SEQUENCE LISTING (64 sequences)

SEQ ID NO: 1 - protein, 122 aa, synthetic construct
  REGION 1..122: note = humanized antibody
QVQLVQSGAEVKKPGASVKVSCKASGYTFTRYYLYWVKQRPGQGLEWIGEINPNNGGTKFNEKFKSRATMTVDKSTRTTYMELSSLRSEDTAVYYCSREDDYDGTPDAMDYWGQGTLVTVSS

SEQ ID NO: 2 - protein, 107 aa, synthetic construct
  REGION 1..107: note = humanized antibody
DIQMTQSPSSLSASVGDRVTITCHASQNINLWLSWYQQKPGKAPKLLIYRASNLHTGVPSRFSGSGSATDFTFTISSLQPEDIATYYCQQGHSYPYTFGGGTKVDIK

SEQ ID NO: 3 - protein, 122 aa, synthetic construct [sequence omitted]

SEQ ID NO: 4 - protein, 107 aa, synthetic construct
  REGION 1..107: note = humanized antibody
DIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIK

SEQ ID NO: 5 - protein, 5 aa, synthetic construct
  REGION 1..5: note = humanized antibody
RYYLY

SEQ ID NO: 6 - protein, 17 aa, synthetic construct
  REGION 1..17: note = humanized antibody
EINPNNGGTKFNEKFKS

SEQ ID NO: 7 - protein, 13 aa, synthetic construct
  REGION 1..13: note = humanized antibody
EDDYDGTPDAMDY

SEQ ID NO: 8 - protein, 11 aa, synthetic construct
  REGION 1..11: note = humanized antibody
HASQNINLWLS

SEQ ID NO: 9 - protein, 7 aa, synthetic construct
  REGION 1..7: note = humanized antibody
RASNLHT

SEQ ID NO: 10 - protein, 9 aa, synthetic construct
  REGION 1..9: note = humanized antibody
QQGHSYPYT

SEQ ID NO: 11 - protein, 5 aa, synthetic construct
  REGION 1..5: note = humanized antibody
RYYMY

SEQ ID NO: 12 - protein, 17 aa, synthetic construct
  REGION 1..17: note = humanized antibody
EINPNNGGTKFNEKFKN

SEQ ID NO: 13 - protein, 13 aa, synthetic construct
  REGION 1..13: note = humanized antibody
EDDYDGTPFAMDY

SEQ ID NO: 14 - protein, 11 aa, synthetic construct
  REGION 1..11: note = humanized antibody
HASQNINVWLS

SEQ ID NO: 15 - protein, 7 aa, synthetic construct
  REGION 1..7: note = humanized antibody
KASNLHT

SEQ ID NO: 16 - protein, 9 aa, synthetic construct
  REGION 1..9: note = humanized antibody
QQGQTYPYT

SEQ ID NO: 17 - protein, 5 aa, synthetic construct
  REGION 1..5: note = humanized antibody
SYLIH

SEQ ID NO: 18 - protein, 17 aa, synthetic construct
  REGION 1..17: note = humanized antibody
VINPRSGDSHYNEKFKD

SEQ ID NO: 19 - protein, 6 aa, synthetic construct
  REGION 1..6: note = humanized antibody
SDYGAY

SEQ ID NO: 20 - protein, 11 aa, synthetic construct
  REGION 1..11: note = humanized antibody
RASQSISNNLH

SEQ ID NO: 21 - protein, 7 aa, synthetic construct
  REGION 1..7: note = humanized antibody
YASQSAF

SEQ ID NO: 22 - protein, 9 aa, synthetic construct
  REGION 1..9: note = humanized antibody
QQSNSWPHT

SEQ ID NO: 23 - protein, 452 aa, synthetic construct
  REGION 1..452: note = humanized antibody
QVQLVQSGAEVKKPGASVKVSCKASGYTFTRYYLYWVKQRPGQGLEWIGEINPNNGGTKFNEKFKSRATMTVDKSTRTTYMELSSLRSEDTAVYYCSREDDYDGTPDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, synthetic construct
  REGION 1..214: note = humanized antibody
DIQMTQSPSSLSASVGDRVTITCHASQNINLWLSWYQQKPGKAPKLLIYRASNLHTGVPSRFSGSGSATDFTFTISSLQPEDIATYYCQQGHSYPYTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 107 aa, synthetic construct
  REGION 1..107: note = humanized antibody
DIQMTQSPSSLSASVGDRVTITCHASQNINVWLSWYQQKPGKAPKLLIYKASNLHTGVPSRFSGSGSGTDFTFTISSLQPEDIATYYCQQGQTYPYTFGQGTKLEIK

SEQ ID NO: 26 - protein, 451 aa, synthetic construct
  REGION 1..451: note = humanized antibody
QVQLVQSGAEVKKPGASVKLSCKASGYIFTRYYMYWVKQRPGQGLEWIGEINPNNGGTKFNEKFKNRATLTVDKSISTAYMELSRLRSDDTAVYYCSREDDYDGTPFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 214 aa, synthetic construct
  REGION 1..214: note = humanized antibody
DIQMTQSPSSLSASVGDRVTITCHASQNINVWLSWYQQKPGKAPKLLIYKASNLHTGVPSRFTGSGSGTDFTFTISSLQPEDIATYYCQQGQTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 366 bp, synthetic construct
  misc_feature 1..366: note = humanized antibody
  source 1..366: mol_type = other DNA
caggtccaactggtgcagtctggggctgaagtgaagaagcctggggcttcagtgaaggtttcctgcaaggcttctggctacaccttcactcggtactatttgtactgggtgaaacagaggcctggacaaggccttgagtggattggagagataaatcctaacaatggtggtactaagttcaatgagaagttcaagagcagagccacaatgactgtagacaaatccacgagaacaacatacatggagctcagcagcctgagatctgaggacacggcggtctattactgttcaagagaggatgattacgacgggacccccgatgctatggactactggggtcaaggaaccctggtcaccgtctcctca

SEQ ID NO: 29 - DNA, 321 bp, synthetic construct
  misc_feature 1..321: note = humanized antibody
  source 1..321: mol_type = other DNA
gacatccagatgacccagtctccatccagtctgtctgcatccgtaggagacagagtcaccatcacttgccatgccagtcagaacattaatctttggttatcttggtaccagcagaaaccaggaaaagcccctaaacttctgatctatagggcttccaacttgcacacaggcgtcccatcaaggtttagtggcagtggatctgcaacagatttcacattcaccatcagcagcctgcagcctgaagacattgccacttactactgtcaacagggtcatagttatccgtacacgttcggaggggggaccaaagtggatatcaaa

SEQ ID NO: 30 - DNA, 1356 bp, synthetic construct
  misc_feature 1..1356: note = humanized antibody
  source 1..1356: mol_type = other DNA
caggtccaactggtgcagtctggggctgaagtgaagaagcctggggcttcagtgaagttgtcctgcaaggcttctggctacatcttcaccagatactatatgtattgggtgaagcagaggcctggacaaggccttgagtggattggagagattaatcctaacaatggtggtactaagttcaatgagaagttcaagaacagggccacactgactgtagacaaatccatcagcacagcatacatggagctcagcaggctgagatctgacgacacggcggtctattattgttcaagagaggatgattacgacgggaccccctttgctatggactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaattcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 31 - DNA, 645 bp, synthetic construct
  misc_feature 1..645: note = humanized antibodies
  source 1..645: mol_type = other DNA
gacatccagatgacccagtctccatccagtctgtctgcatccgtaggagacagagtcaccatcacttgccatgccagtcagaacattaatgtttggttatcttggtaccagcagaaaccaggaaaagcccctaaactcttgatctataaggcttccaacttgcacacaggcgtcccatcaagatttactggcagtggatctggaacagatttcacattcaccatcagcagcctgcagcctgaagacattgccacttactactgtcaacagggtcaaacttatccatacacgttcggacaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 32 - protein, 513 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 33 - protein, 334 aa, Homo sapiens
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSSDTNKSA

SEQ ID NO: 34 - protein, 584 aa, Homo sapiens
MKMASSLAFLLLNFHVSLFLVQLLTPCSAQFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQPWIAALAGTLPISLLLLAGASYFLWRQQKEKIALSRETEREREMKEMGYAATEQEISLREKLQEELKWRKIQYMARGEKSLAYHEWKMALFKPADVILDPDTANAILLVSEDQRSVQRAEEPRDLPDNPERFEWRYCVLGCENFTSGRHYWEVEVGDRKEWHIGVCSKNVERKKGWVKMTPENGYWTMGLTDGNKYRALTEPRTNLKLPEPPRKVGIFLDYETGEISFYNATDGSHIYTFPHASFSEPLYPVFRILTLEPTALTICPIPKEVESSPDPDLVPDHSLETPLTPGLANESGEPQAEVTSLLLPAHPGAEVSPSATTNQNHKLQARTEALY

SEQ ID NO: 35 - DNA, 20 bp, Homo sapiens
  source 1..20: mol_type = genomic DNA
ggaaatcgtgcgtgacatta

SEQ ID NO: 36 - DNA, 19 bp, Homo sapiens
  source 1..19: mol_type = genomic DNA
aggaggaaggctggaagag

SEQ ID NO: 37 - DNA, 20 bp, Homo sapiens
  source 1..20: mol_type = genomic DNA
ttccaggtcatagtgtctgc

SEQ ID NO: 38 - DNA, 19 bp, Homo sapiens
  source 1..19: mol_type = genomic DNA
tgagcagctgagcaaaagg

SEQ ID NO: 39 - DNA, 16 bp, Homo sapiens
  source 1..16: mol_type = genomic DNA
tgggaataccaaggga

SEQ ID NO: 40 - DNA, 20 bp, Homo sapiens
  source 1..20: mol_type = genomic DNA
agtgagcagctggaccaaga

SEQ ID NO: 41 - DNA, 21 bp, Homo sapiens
  source 1..21: mol_type = genomic DNA
gagggaatactaagaaatggt

SEQ ID NO: 42 - DNA, 20 bp, Homo sapiens
  source 1..20: mol_type = genomic DNA
gaagagggagacatgaaagt

SEQ ID NO: 43 - DNA, 23 bp, Homo sapiens
  source 1..23: mol_type = genomic DNA
gcactatttttagccggaacctt

SEQ ID NO: 44 - DNA, 24 bp, Homo sapiens
  source 1..24: mol_type = genomic DNA
ttgaggagaaaaactggattgaga

SEQ ID NO: 45 - DNA, 23 bp, Homo sapiens
  source 1..23: mol_type = genomic DNA
aggagaagaggctgaggaacaag

SEQ ID NO: 46 - DNA, 24 bp, Homo sapiens
  source 1..24: mol_type = genomic DNA
gagggagagaagcaactacagacc

SEQ ID NO: 47 - DNA, 22 bp, Homo sapiens
  source 1..22: mol_type = genomic DNA
cagcacctctcaagcagaaaac

SEQ ID NO: 48 - DNA, 22 bp, Homo sapiens
  source 1..22: mol_type = genomic DNA
gttgggcattggtgtagacaac

SEQ ID NO: 49 - DNA, 22 bp, Homo sapiens
  source 1..22: mol_type = genomic DNA
ccaggagaagattccaaagatg

SEQ ID NO: 50 - DNA, 22 bp, Homo sapiens
  source 1..22: mol_type = genomic DNA
ggaaggttcaggttgttttctg

SEQ ID NO: 51 - DNA, 23 bp, Homo sapiens
  source 1..23: mol_type = genomic DNA
gttttgggttctcttggctgtta

SEQ ID NO: 52 - DNA, 23 bp, Homo sapiens
  source 1..23: mol_type = genomic DNA
acactcttttggatgctctggtc

SEQ ID NO: 53 - DNA, 22 bp, Homo sapiens
  source 1..22: mol_type = genomic DNA
tcccatcttccaagggtactaa

SEQ ID NO: 54 - DNA, 22 bp, Homo sapiens
  source 1..22: mol_type = genomic DNA
ggtagccactgaaagaatttgg

SEQ ID NO: 55 - DNA, 23 bp, Homo sapiens
  source 1..23: mol_type = genomic DNA
gggggttgaggtatcagaggtaa

SEQ ID NO: 56 - DNA, 23 bp, Homo sapiens
  source 1..23: mol_type = genomic DNA
gctccaagagaaaggcatctaca

SEQ ID NO: 57 - DNA, 21 bp, Homo sapiens
  source 1..21: mol_type = genomic DNA
gacatcaaaagataaccactc

SEQ ID NO: 58 - DNA, 21 bp, Homo sapiens
  source 1..21: mol_type = genomic DNA
tctatgacaagttcaagcaga

SEQ ID NO: 59 - DNA, 21 bp, Homo sapiens
  source 1..21: mol_type = genomic DNA
tctatcaccagacttgacaca

SEQ ID NO: 60 - DNA, 23 bp, Homo sapiens
  source 1..23: mol_type = genomic DNA
cctaatcactctcctcctcttcc

SEQ ID NO: 61 - DNA, 23 bp, Homo sapiens
  source 1..23: mol_type = genomic DNA
cggtctctgtgatttgtaaccag

SEQ ID NO: 62 - DNA, 22 bp, Homo sapiens
  source 1..22: mol_type = genomic DNA
tactatgctttccccatccatc

SEQ ID NO: 63 - protein, 115 aa, synthetic construct
  REGION 1..115: note = humanized antibody
QVQMQQSGAEVKKPGASVKVSCKASGYAFTSYLIHWIKQRPGQGLEWIGVINPRSGDSHYNEKFKDRVTMTADQSISTAYMELSRLRSDDTAVYYCARSDYGAYWGQGTLVTVSS

SEQ ID NO: 64 - protein, 107 aa, synthetic construct
  REGION 1..107: note = humanized antibody
EIVLTQSPATLSVSPGERATLSCRASQSISNNLHWYQQKPGQAPRLLIKYASQSAFGIPARFSGSGSGTEFTLTISSLQSEDFAVYFCQQSNSWPHTFGQGTKLEIK

(19) United States
(12) Patent Application Publication
(10) Pub. No.: US 2025/0129165 A1
(43) Pub. Date: Apr. 24, 2025

(54) BUTYROPHILIN (BTN) 3A ACTIVATING ANTIBODIES FOR USE IN METHODS FOR TREATING INFECTIOUS DISORDERS

(71) Applicants: IMCHECK THERAPEUTICS, Marseille (FR); INSERM (INSTITUT NATIONAL DE LA SANTÉ ET DE LA RECHERCHE MÉDICALE), Paris (FR); UNIVERSITÉ D'AIX-MARSEILLE, Marseille (FR); CENTRE NATIONAL DE LA RECHERCHE SCIENTIFIQUE, Paris (FR); INSTITUT JEAN PAOLI & IRENE CALMETTES, Marseille (FR); ASSISTANCE PUBLIQUE HOPITAUX DE MARSEILLE, Marseille (FR); INSTITUT DE RECHERCHE POUR LE DÉVELOPPEMENT, Marseille 2 (FR)

(72) Inventors: Daniel OLIVE, Marseille (FR); Soraya MEZOUAR, Marseille (FR); Jean-Louis MEGE, Marseille (FR); Paul FROHNA, Marseille Cedex 09 (FR); Alemseged TRUNEH, Sudbury, MA (US); Laetitia GAY, Marseille Cedex 09 (FR)

(73) Assignees: IMCHECK THERAPEUTICS, Marseille (FR); INSERM (INSTITUT NATIONAL DE LA SANTÉ ET DE LA RECHERCHE MÉDICALE), Paris (FR); UNIVERSITÉ D'AIX-MARSEILLE, Marseille (FR); CENTRE NATIONAL DE LA RECHERCHE SCIENTIFIQUE, Paris (FR); INSTITUT JEAN PAOLI & IRENE CALMETTES, Marseille (FR); ASSISTANCE PUBLIQUE HOPITAUX DE MARSEILLE, Marseille (FR); INSTITUT DE RECHERCHE POUR LE DÉVELOPPEMENT, Marseille 2 (FR)

(21) Appl. No.: 18/705,452

(22) PCT Filed: Oct. 27, 2022
(86) PCT No.: PCT/EP2022/080048

§ 371 Date: Apr. 26, 2024

(30) Foreign Application Priority Data
Oct. 27, 2021 (EP) 21306505.5

(51) Int. Cl. C07K 16/28; A61K 45/06; A61P 31/04; A61P 31/14

(52) U.S. Cl. CPC C07K 16/2827; A61K 45/06; A61P 31/04; A61P 31/14; C07K 2317/565; C07K 2317/71; C07K 2317/734; C07K 2317/75; C07K 2317/92

(57) ABSTRACT

The present disclosure relates to methods for treating infectious disorders. In particular, the disclosure provides BTN3A activating antibodies, and their use in treating infectious disorders in a human subject in need thereof, such as disorders caused by SARS-Cov2 or Coxiella burnetii infection.

The present disclosure relates to methods for treating infectious disorders. In particular, the disclosure provides BTN3A activating antibodies, and their use in treating infectious disorders in a human subject in need thereof, such as disorders caused by SARS-Cov2 or Coxiella burnetii infection.

### BACKGROUND

The global pandemic of coronavirus disease 2019 caused by the emerging severe acute respiratory syndrome coronavirus-2 (SARS-CoV-2) has resulted to date in more than 220 million global cases with more than 4.8 million deaths. While some infected persons remain asymptomatic, some will develop a mild respiratory disease that will resolve with no or little medical attention, and others (10 to 20% of symptomatic persons), will experience a severe disease often occurring as a sudden deterioration ˜11 days after the onset of first symptoms (Chen et al., 2020), associated with respiratory failure and multi-organ complications possibly leading to patients' deaths (Gupta et al., 2020; Schultze and Aschenbrenner, 2021). While vaccination remains an asset against the virus and its variants to prevent the disease, only symptomatic treatment is currently provided. In this context, efforts must be continued in the fight against the disease with the need to develop drug treatments. In another context, immunotherapy could be considered in the case of Q fever. This worldwide zoonosis is caused by the bacteria Coxiella burnetii and leads in humans to a primary infection (acute Q fever) that is symptomatic in 40% of cases. A progression to a focal persistent infection is also observed in 1 to 5% of cases, with mainly cardiovascular and osteoarticular manifestations. Currently, the main treatment for acute Q fever is doxycycline. In some cases, such as endocarditis, prolonged treatment for several months with doxycycline and hydroxychloroquine is prescribed.

Current clinical management of COVID-19 consists of prevention, control measures such as social distancing, and supportive care including supplemental oxygen and mechanical ventilatory support when indicated. To date, FDA has approved one drug, remdesivir (Veklury), for the treatment of COVID-19 in hospitalized patients aged 12 years and older who weigh at least 40 kg.

Human Vγ9Vδ2 T cells, that represent 2-5% of peripheral blood T cells, are found expanded during infection by a wide range of microbial agents up to occupied more than 50% of the circulating T cell pool (Chen Z W. Cell Mol Life Sci 2011; 68:2409-1), as reported for patients with mycobacterial disease, listeriosis, salmonellosis, brucellosis, tularemia, legionellosis and Q fever (Morita et al Immunol Rev 2007; 215:59-76; Balbi et al. Am Rev Respir Dis 1993; 148:1685-90; Jouen-Beades F, et al. Infect Immun 1997; 65:4267-72; Hara T, Mizuno Y, Takaki K, et al. J Clin Invest 1992; 90:204-10; Bertotto A, Gerli R, Spinozzi F, et al. Eur J Immunol 1993; 23:1177-80; Poquet Y, Kroca M, Halary F, et al. Infect Immun 1998; 66:2107-14; Kroca M, Johansson A, Sjöstedt A, Tärnvik A. Clin Diagn Lab Immunol 2001; 8:949-54; Schneider T, Jahn H-U, Liesenfeld O, et al. Clin Infect Dis 1997; 24:261-4). Furthermore, local expansion of Vγ9Vδ2 T cells have also been reported in the bronchoalveolar lavage fluid from patients with active pulmonary tuberculosis and in cerebral spinal fluid from patients with bacterial meningitis (Chen Z W, Letvin N L. Microbes Infect 2003; 5:491-8; Dieli F, Sireci G, Di Sano C, et al. Mol Med 1999; 5:301-12; Caccamo N, La Mendola C, Orlando V, et al. Blood 2011; 118:129-38).

Two direct antimicrobial actions of Vγ9Vδ2 T cells against various viruses, protozoa and bacteria were reported, including cytotoxic activity to pathogen-infected cells and a cell-mediated non-cytolytic activity based on cytokine production (Bonneville M, Scotet E. Curr Opin Immunol 2006; 18:539-46; Chen Z W. Cell Mol Immunol 2013; 10:58-64; Poccia F, Agrati C, Martini F, Capobianchi M R, Wallace M, Malkovsky M. Microbes Infect 2005; 7:518-28; Dong P, Ju X, Yan Y, et al. Front Immunol 2018; 9. DOI: 10.3389/fimmu.2018.02812). In vitro studies have shown that γT cells are able to effectively kill intracellular pathogens such as M. tuberculosis, L. monocytogenes, and Brucella suis. Ryan-Payseur B, Frencher J, Shen L, Chen C Y, Huang D, Chen Z W. J Immunol 2012; 189:1285-93; Spencer C T, Abate G, Sakala I G, et al. PLOS Pathog 2013; 9: e1003119; Dieli F, Troye-Blomberg M, Ivanyi J, et al. J Infect Dis 2001; 184:1082-5; Martino A, Casetti R, Sacchi A, Poccia F. J Immunol Baltim Md 1950 2007; 179:3057-64; Oliaro J, Dudal S, Liautard J, Andrault J-B, Liautard J-P, Lafont V. J Leukoc Biol 2005; 77:652-60.

Several evidence highlight the key role of Vγ9Vδ2 T cells in Q fever, an infectious disease caused by the intracellular bacterium Coxiella burnetii. (1) During the acute phase of the disease, number and proportion of Vγ9Vδ2 T cells were found increased (2) with a significant expression of the activation marker HLA-DR but not CD25. Schneider T, Jahn H-U, Liesenfeld O, et al. Clin Infect Dis 1997; 24:261-4.

WO2012/080351 reports BTN3A activating antibodies, such as murine mAb 7.2 or mAb 20.1 having the capacity to induce the proliferation and cytokine secretion of Vγ9Vδ2 T cells.

WO2020/025703 further reports specific humanized BTN3A activating antibodies, in particular for their use in treating cancer disorders.

WO2020/136218 also discloses fragments derived from Fab fragment of an anti-BTN3A antibody mAb103.2 and their use as BTN3A activating antibody for inducing the proliferation and cytokine secretion of Vγ9Vδ2 T cells

However, to the knowledge of the inventors, there is no evidence of a plausible use of an activating compound of Vγ9Vδ2 T cells, and in particular, BTN3A activating antibodies, as for treating infectious disorders caused by Coxiella burnetii or SARS-Cov2.

The inventors have now surprisingly found that treatments with BTN3A activating antibodies such as treatment with mAb 20.1 enhances Vγ9Vδ2 T cell responses against SARS-CoV-2 and Coxiella burnetii infected cells in particular to diminish viral and bacterial load, respectively.

### SUMMARY

A first aspect of the disclosure relates to BTN3A activating antibody, for use in treating infectious disorders in a human subject in need thereof, in particular for treating disorder caused by SARS-Cov2 infection or Coxiella Burnetii infection.

In specific embodiments, said BTN3A activating antibody is mAb 20.1 or a humanized form of mAb 20.1.

Another aspect of the disclosure relates to novel humanized forms of mAb 20.1 or their pharmaceutical compositions, in particular for their use in treating disorders caused by SARS-Cov2 infection or Coxiella Burnetii infection.

Other specific aspects and specific embodiments are disclosed hereafter.

### LEGENDS OF THE FIGURES

FIG. 1. Expression BTN2A and BTN3A in response to SARS-CoV-2-infection in monocytes, MDMs and lung epithelial cell lines

(A, B) Monocytes, MDMs, BEAS-2B and MRC-5 cells were stimulated with SARS-CoV-2 IHU-MI6 strain (1 MOI) for 24 hours. Mean fluorescence intensity (MFI) of BTN2A (A) and BTN3A (B) expression was investigated on healthy donor monocytes and MDMs, as well as lung epithelial cell lines BEAS-2B and MRC-5 (n=3). The gene expression of the two BTN2A isoforms (A1, A2) (A) and the three BTN3A isoforms (A1, A2, A) (B) was investigated by qRT-PCR after normalization with housekeeping gene as endogenous control. Relative expression of investigated genes at 24 hours of stimulation was evaluated for monocytes and MDMs (n=6), and BEAS-2B and MRC-5 cells (n=3).

FIG. 2. Impact of SARS-CoV-2 on Vγ9Vδ2 T cells viability. Vγ9Vδ2 T cells (isolated from 3 healthy volunteers) were stimulated with SARS-CoV-2 IHU-MI6 strain (0.25, 0.5 or 1 MOI). After 24 hours, the viability of Vγ9Vδ2 T cells was evaluated by flow cytometry as the percentage of live cells in the Vγ9Vδ2 T cell population. Values represent mean±standard error of the mean.

FIG. 3. Assessment of Vγ9Vδ2 T lymphocytes anti-SARS-CoV-2 responses. (A, B) Monocytes, MDMs, BEAS-2B and MRC-5 cells (n=6) were stimulated with SARS-CoV-2 IHU-MI6 strain (1 MOI) and co-cultured with Vγ9Vδ2 T cells at effector-to-target (E:T) ratio of 1:1 for 24 hours in presence of anti-BTN3A 20.1 Ab-(0, 0.1, 1 or 10 μg/ml). (A) After 24 hours, SARS-CoV-2 viral load was quantitated by RT-PCR. Data were analyzed by the following formula 2^{\hat{ }-(Ct anti-BTN3A ICT01-Ct diluent)} and represented in % viral copies. The cytotoxicity was assessed by flow cytometry as the percentage of Caspase 3/7⁺ cells in the target cell population. (B) The culture supernatants from co-cultures were analyzed for the presence of IFN-γ by ELISA. Values represent mean±standard error. *p<0.05, **p<0.01, ***p<0.001 and ****p<0.0001.

FIG. 4. C. burnetii infection modulates BTN3A and BTN2A expression. Monocytes isolated from healthy donors (n=4) were infected with C. burnetii strains (50 MOI) for 24 hours. (A) The relative gene expression of BTN3A isoforms (A1, A2, A3) and (B) the BTN3A protein expression were investigated by qRT-PCR and flow cytometry, respectively. (D) The relative gene expression of BTN2A isoforms (A1, A2) and (E) the BTN2A protein expression were investigated by qRT-PCR and flow cytometry, respectively. (C) BTN3A and (F) BTN2A protein expression was investigated for PBMCs from Q fever patients (n=6) or healthy donors (n=3). Values represent mean±standard error. *p<0.05, **p<0.01, ***p<0.001 and ****p<0.0001.

FIG. 5. Involvement of BTN3A and BTN2A in C. burnetii infection. CRISPR-Cas9-mediated inactivation of BTN3A or BTN2A was performed in THP-1 cell line. THP-1 cells transduced with a guide targeting all BTN2A isoforms (BTN2AKO) or all BTN3A isoforms (BTN3AKO) or with an irrelevant CRISPR guide (mock) for control cells were infected with C. burnetii NM1 (50 MOI) (n=3). (A) After 4 and 24 hours of infection, the number of bacterial DNA copies within THP-1 cells was assessed by qPCR. (B) THP-1 cells were incubated with C. burnetii for 4 h (day 0), then washed to eliminate free bacteria and incubated for 4 days. Each day, the number of bacterial DNA copies was evaluated by qPCR.

FIG. 6. Involvement of BTN3A and BTN2A in the inflammatory response to C. burnetii infection. THP-1 cells transduced with an irrelevant CRISPR guide (mock) or a guide targeting all BTN2A isoforms (BTN2AKO) or all BTN3A isoforms (BTN3AKO) were infected with C. burnetii NM1 (100 MOI) (n=3). After 24 hours infection, the expression of genes involved in the inflammatory (TNF, IL1B, IL6, IFNG, CXCL10) or immunoregulatory (IL10, TGFB1, IL1RA, CD163) response was investigated by quantitative reverse-transcription polymerase chain reaction after normalization with housekeeping actin gene as endogenous control. Data are illustrated as (A) relative expression (RQ) of investigated genes. (B) After 24 hours infection, TNF-α, IL-1β, IFN-γ, IL-6, IL-10, and TGF-β release were evaluated in the culture supernatants by ELISA assay. Values represent mean±standard error. *p<0.05 and **p<0.01.

FIG. 7. Infection with C. burnetii leads BTN3A and BTN2-dependent activation of Vγ9Vδ2 T lymphocytes. (A) Monocytes isolated from healthy donors (n=3) previously infected 24 hours with C. burnetii NM1 (50 or 100 MOI) were co-cultured with Vγ9Vδ2 T cells expanded from healthy donor (E:T ratio of 1:1). Vγ9Vδ2 T cell degranulation (% CD107ab+ cells) was assessed after 4 hours of co-culture by flow cytometry. (B, C, F) Monocytes isolated from healthy donors (n=4) previously infected 24 hours with C. burnetii strains (50 MOI) were co-cultured with Vγ9Vδ2 T cells expanded from healthy donor (E:T ratio of 1:1) in the presence of (B) anti-BTN2A (clone 7.48), (C) anti-BTN3A (clone 103.2) or (F) anti-BTN3A (clone 20.1) antibody (10 μg/ml). Vγ9Vδ2 T cell degranulation (% CD107ab+ cells) was assessed after 4 hours of co-culture by flow cytometry. (G) The cytotoxicity was assessed by flow cytometry as the percentage of Caspase 3/7⁺ cells in the target cell population after 4 hours of co-culture in presence of the reference anti-BTN3A 20.1 antibody (10 μg/ml). (D, E, H) Vγ9Vδ2 T cells expanded from healthy donor were co-cultured with PBMCs from Q fever patients (n=6) or healthy donors (n=3) (E:T ratio of 1:1) in the presence of (D) anti-BTN2A (clone 7.48), (E) anti-BTN3A (clone 103.2) or (F) anti-BTN3A (clone 20.1) antibodies (10 μg/ml). Vγ9Vδ2 T cell degranulation (% CD107ab+ cells) was assessed after 4 hours of co-culture by flow cytometry. Values represent mean±standard error. *p<0.05, **p<0.01, ***p<0.001 and ****p<0.0001.

FIG. 8. The reference 20.1 BTN3A activating antibody increases antimicrobial activity of Vγ9Vδ2 T cells towards C. burnetii infected monocytes. (A, B) Monocytes isolated from healthy donors (n=4) previously infected 24 hours with C. burnetii NM1 (50 MOI) were co-cultured with Vγ9Vδ2 T cells expanded from healthy donor (E:T ratio of 1:1) in the presence of anti-BTN3A 20.1 antibody (0-10 μg/ml). After 4 hours of co-culture, C. burnetii load was measured by (A) flow cytometry and (B) qPCR. Values represent mean±standard error. *p<0.05, **p<0.01 and ***p<0.001.

FIG. 9. The reference 20.1 BTN3A activating antibody increases the secretion of cytokines and cytotoxic molecules in Vγ9Vδ2 T cell/infected-monocyte co-cultures. Monocytes isolated from healthy donors (n=4) previously infected 24 hours with C. burnetii NM1 (50 MOI) were co-cultured with Vγ9Vδ2 T cells expanded from healthy donor (ET ratio of 1:1) in the presence of anti-BTN3A antibody (clone 20.1) (0-10 μg/ml). After 4 hours of co-culture, the culture supernatants were analyzed for the presence of cytokines (A, left panel) and cytotoxic molecules (B, right panel) by ELISA assay. Values represent mean±standard error. *p<0.05, **p<0.01, ***p<0.001 and ****p<0.0001.

### DETAILED DESCRIPTION

### Definitions

In order that the present disclosure may be more readily understood, certain terms are first defined. Additional definitions are set forth throughout the detailed description.

The term “agonist” is used herein for a molecule (e.g., a small organic molecule or a polypeptide, such as an antibody) that binds to a receptor and activates the receptor to produce a biological response. A selective agonist is selective for a specific type of receptor. Binding to the receptors can be, for example, specific binding as determined by surface plasmon resonance at biologically relevant concentrations.

The terms “polypeptide”, “protein” or “peptide” as used herein refer to any chain of amino acid residues, regardless of its length or post-translational modification (such as glycosylation).

As used herein, the term “BTN3A” has its general meaning in the art. In specific embodiments, it refers to human BTN3A polypeptides including either BTN3A1 of SEQ ID NO:32, BTN3A2 of SEQ ID NO:33 or BTN3A3 of SEQ ID NO:34.

The term “antibody” as used herein refers to immunoglobulin molecules and immunologically active portions of immunoglobulin molecules, i.e., molecules that contain an antigen binding site that immunospecifically binds an antigen. The term “antibody” or “immunoglobulin” have the same meaning and will be used equally in the present disclosure. As such, the term antibody encompasses not only whole antibody molecules, but also antibody fragments as well as variants (including derivatives) of antibodies. The term “antibody” as used herein also includes bispecific or multispecific molecules. An antibody can be derivatized or linked to another functional molecule, e.g., another peptide or protein (e.g., another antibody or ligand for a receptor) to generate a bispecific molecule that binds to at least two different binding sites or target molecules. The antibody may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule, an antibody of the disclosure can be functionally linked (e.g., by chemical coupling, genetic fusion, noncovalent association or otherwise) to one or more other binding molecules, such as another antibody, antibody fragment, peptide or binding mimetic, such that a bispecific molecule result. Additionally, for the embodiment in which the bispecific molecule is multi-specific, the molecule can further include a third binding specificity, in addition to the first and second target epitope.

In natural antibodies of rodents and primates, two heavy chains are linked to each other by disulfide bonds, and each heavy chain is linked to a light chain by a disulfide bond. There are two types of light chains, lambda (λ) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate in the antibody binding site, or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

The non-CDR regions of a mammalian antibody may be replaced with similar regions of conspecific or heterospecific antibodies while usually retaining the epitopic specificity of the original antibody. This is most clearly manifested in the development and use of “humanized” antibodies in which non-human CDRs are covalently joined to human FR and/or Fc/pFc′ regions to produce a functional antibody.

As used herein, “humanized” describes antibodies wherein some, most or all of the amino acids outside the CDR regions are replaced with corresponding amino acids derived from human immunoglobulin molecules in order to reduce immunogenicity in human subject. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3 Å of the CDRs in a three-dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization. In some humanized forms of antibodies, some, most or all of the amino acids outside the CDR regions can be replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgGI, IgG2, IgG3, IgG4, IgA and IgM molecules. A “humanized” antibody normally retains a similar antigenic specificity as the original antibody. However, using certain methods of humanization, the affinity and/or specificity of binding of the antibody may be increased using methods of “directed evolution”, as described by Wu et al., Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for selecting human antibodies from human antibody libraries. These include phage display technology (U.S. Pat. Nos. 5,565,332 and 5,573,905) or in vitro stimulation of human B cells (U.S. Pat. Nos. 5,229,275 and 5,567,610). The contents of these patents are incorporated herein by reference.

The term “antigen-binding fragment” of an antibody (or simply “antibody fragment”), as used herein, refers to full length or to one or more fragments of an antibody that retain the ability to specifically bind to an antigen (e.g., a BTN3A protein as above defined) as well as their BTN3A activating properties. In specific embodiments, a BTN3A activating antibody for use in treating infectious disorders as disclosed herein is an antibody fragment, and more particularly any protein including an antigen-binding domain of a BTN3A activating antibody as disclosed herein. Well known-antibody fragments comprise: a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain, or any fusion proteins comprising such antigen-binding fragments; a diabody, which refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Furthermore, although the two domains of the Fv fragment, VL and VH, are coded for by separate genes, they can be joined, using recombinant methods, by a synthetic linker that enables them to be made as a single chain protein in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding fragment” of an antibody (also shortly named herein antibody fragment). More generally antibody fragments as herein intended also encompass single-domain antibodies that are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1). These antibody fragments are obtained using conventional techniques known to those of skill in the art, and the fragments are screened for utility in the same manner as are intact antibodies. Well-suited antibody fragments include, but are not limited to, Fv, Fab, F(ab′)2, Fab′, dsFv, scFv, sc(Fv)2 and diabodies. Antibody fragments can be made by various techniques, including but not limited to proteolytic digestion of an intact antibody as well as production by recombinant host cells as described herein.

The term “monoclonal antibody” as used herein refers to a preparation of antibody molecules of single specificity. A monoclonal antibody displays a single binding specificity and affinity for a particular epitope. Accordingly, the term “human monoclonal antibody” refers to an antibody displaying a single binding specificity which has variable and constant regions derived from or based on human germline immunoglobulin sequences or derived from completely synthetic sequences. The method of preparing the monoclonal antibody is not relevant for the binding specificity.

“Recombinant antibodies” are antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies. In some embodiments, a recombinant human antibody for use according to this disclosure has the same amino acid sequence as the corresponding naturally occurring human antibody but differs structurally from said naturally occurring human antibody. For example, in some embodiments the glycosylation pattern is different as a result of the recombinant production of the recombinant human antibody. In some embodiments the recombinant human antibody is chemically modified by addition or subtraction of at least one covalent chemical bond relative to the structure of the human antibody that occurs naturally in humans.

An “isolated antibody”, as used herein, refers to an antibody that is substantially free of other antibodies having different antigenic specificities (e.g., an isolated antibody that specifically binds to BTN3A is substantially free of antibodies that specifically bind to other antigens than BTN3A). An isolated antibody that specifically binds to BTN3A may, however, have cross-reactivity to other antigens, such as related BTN3A molecules from other species. Moreover, an isolated antibody may be substantially free of other cellular material and/or chemicals.

The phrases “an antibody recognizing an antigen” and “an antibody having specificity for an antigen” are used interchangeably herein with the term “an antibody which binds specifically to an antigen”. The terms “an anti-BTN3A antibody” or “a BTN3A antibody” are also shortly used herein with the meaning of “an antibody recognizing BTN3A”.

As used herein, the term “activating antibody” refers to an antibody able to directly or indirectly induce immune functions of effector cells. In particular, as used herein, a BTN3A activating antibody has at least the capacity to induce the activation of γδ T cells, typically Vγ9Vδ2 T cells, in co-culture with BTN3A expressing cells, with an EC50 below 5 μg/ml, preferably of 1 μg/ml or below, as measured in a degranulation assay (such degranulation assay is described in the Examples below).

As used herein, the term “binding” in the context of the binding of an antibody to a predetermined antigen or epitope, notably BTN3A, means typically a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less when determined by for instance surface plasmon resonance (SPR) technology in a BIAcore instrument using typically a soluble form of the antigen as the ligand and the antibody as the analyte. BIACORE® (GE Healthcare, Piscataway, NJ) is one of a variety of surface plasmon resonance assay formats that are routinely used to epitope bin panels of monoclonal antibodies. Typically, an antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein), which is not identical or closely related to the predetermined antigen. When the K_{D} of the antibody is very low (that is, the antibody has a high affinity), then the K_{D} with which it binds the antigen is typically at least 10,000-fold lower than its K_{D} for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

The term “affinity”, as used herein in the context of an antibody, means the strength of the binding of an antibody to an epitope.

The term “Kon” or “Kass” (Ka), as used herein, is intended to refer to the association rate of a particular antibody-antigen interaction, whereas the term “Kdis” (Kd) or “Koff,” as used herein, is intended to refer to the dissociation rate of a particular antibody-antigen interaction.

The term “K_{D}”, as used herein, is intended to refer to the equilibrium dissociation constant, which is obtained from the ratio of k_{off} to kₒₙ (i.e. koff/kon) and is expressed as a molar concentration (M). The K_{D} value relates to the concentration of antibody (the amount of antibody needed for a particular experiment) and so the lower the K_{D} value (lower concentration) and thus the higher the affinity of the antibody. K_{D} values for antibodies can be determined using methods well established in the art. Preferred methods for determining the K_{D} values of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. A method for determining the K_{D} of an antibody is by using surface plasmon resonance, or by using a biosensor system such as a Biacore® (see also for detailed information regarding affinity assessment Rich R L, Day Y S, Morton T A, Myszka D G. High-resolution and high-throughput protocols for measuring drug/human serum albumin interactions using BIACORE®. Anal Biochem. 2001 Sep. 15; 296 (2): 197-207) or Octet® systems. The Octet® platform is based on bio-layer interferometry (BLI) technology. The principle of BLI technology is based on the optical interference pattern of white light reflected from two surfaces-a layer of immobilized protein and an internal reference layer. The binding between a ligand immobilized on the biosensor tip surface and an analyte in solution produces an increase in optical thickness at the biosensor tip, which results in a shift in the interference pattern measured in nanometers. The wavelength shift (AA) is a direct measure of the change in optical thickness of the biological layer, when this shift is measured over a period of time and its magnitude plotted as a function of time, a classic association/dissociation curve is obtained. This interaction is measured in real-time, allowing to monitor binding specificity, association rate and dissociation rate, and concentration. (see Abdiche et al. 2008). Affinity measurements are typically performed at 25° C.

As used herein, the term “specificity” refers to the ability of an antibody to detectably bind an epitope presented on an antigen, such as a BTN3A. In some embodiments, it is intended to refer to an antibody that binds to human BTN3A as expressed on peripheral blood marrow cells (PBMCs), preferably with an EC50 below 50 μg/ml and more preferably below 10 μg/ml as determined by flow cytometry as described in the Examples. In other embodiments, it binds to an antigen recombinant polypeptide with a K_{D} of 100 nM or less, 10 nM or less, 1 nM or less, 100 pM or less, or 10 pM or less, as measured by SPR measurements as described in the Examples).

An antibody that “cross-reacts with an antigen other than BTN3A” is intended to refer to an antibody that binds that antigen other than BTN3A with a K_{D} of 10 nM or less, 1 nM or less, or 100 pM or less. An antibody that “does not cross-react with a particular antigen” is intended to refer to an antibody that binds to that antigen, with a K_{D} of 1 UM or greater, or a K_{D} of 10 UM or greater. In certain embodiments, such antibodies that do not cross-react with the antigen exhibit essentially undetectable binding against these proteins in standard binding assays.

Specificity can further be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a BTN3A polypeptide).

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

As used herein, the term, “optimized” means that a nucleotide sequence has been altered to encode an amino acid sequence using codons that are preferred in the production cell or organism, generally a eukaryotic cell, for example, a Chinese Hamster Ovary cell (CHO) or a human cell. The optimized nucleotide sequence is engineered to retain completely or as much as possible the amino acid sequence originally encoded by the starting nucleotide sequence. The amino acid sequences encoded by optimized nucleotide sequences are also referred to as optimized.

The term “identity” as used herein in reference to polypeptide sequences, refers to the amino acid sequence identity between two molecules. When an amino acid position in both molecules is occupied by the same amino acid, then the molecules are identical at that position. The identity between two polypeptides is a direct function of the number of identical positions. In general, the sequences are aligned so that the highest order match is obtained (including gaps if necessary). The percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. Alternatively, the percent identity between two amino acid sequences can be determined using published techniques and widely available computer programs, such as BLASTP, FASTA (Atschul et al., J. Molecular Biol. 215:403, 1990), or the Needleman and Wunsch (J. Mol, Biol. 48:444-453, 1970) algorithm which has been incorporated into the GAP program in the GCG software package (Devereux et al., Nucleic Acids Res. 12:387, 1984, typically available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

Generally, but not necessarily, it is preferable for amino acid substitutions relative to the reference polypeptide such as CDR regions to be conservative amino acid substitutions.

As used herein, “conservative amino acid substitution” means a given amino acid can be replaced by a residue having similar physiochemical characteristics, e.g., substituting one aliphatic residue for another (such as 11e, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Gly (G), Ala (A), Val (V), Leu (L), Ile (I), Pro (P), Phe (F), Trp (W), Met (M); (2) uncharged polar: Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

The percent identity between two nucleotide amino acid sequences may also be determined using for example algorithms such as the BLASTN program for nucleic acid sequences using as defaults a word length (W) of 11, an expectation (E) of 10, M=5, N=4, and a comparison of both strands.

### Activating BTN3A Antibody for Use of the Present Disclosure

The present disclosure relates to the use of BTN3A activating antibody in methods for treating infectious disorders in a subject in need thereof, more specifically a disorder caused by Coxiella burnetii infection, e.g. Q fever, or a disorder caused by SARS-Cov2, e.g. COVID-19.

In specific embodiments, a BTN3A activating antibody for use according to the present disclosure exhibits the following properties:
(i) it binds to BTN3A with a K_{D} of 10 nM or less, preferably with a K_{D} of 1 nM or less, as measured by SPR, for example as described in the Examples;
(ii) it binds to human PBMCs with an EC₅₀ of 50 μg/ml or below, preferably of 10 μg/ml or below, as measured in a flow cytometry assay, for example as described in the Examples; and,
(iii) it induces the activation of γδ T cells, typically Vγ9Vδ2 T cells, in co-culture with BTN3 expressing cells, with an EC₅₀ below 5 μg/ml, preferably of 1 μg/ml or below, as measured with a degranulation assay, for example as described in the Examples.

In certain embodiments that may be combined with the following specific embodiments, a BTN3A activating antibody for use of the present disclosure is an antibody fragment of the specific antibodies as disclosed below. Antibody fragments include for example, but are not limited to, Fab, Fab′, Fab′-SH, F(ab′)2, Fv, Unibody, and scFv fragments, diabodies, single domain or nanobodies and other fragments. Preferably, it is a monovalent antibody, such as a Fab of scFv fragments. In particular, it is a monovalent BTN3A activating antibody such as a Fab fragment having all 6 CDRs of mAb 103.2.

In some embodiments, antibodies for use of the present disclosure are chimeric, humanized, or human antibodies. In preferred embodiments of the present disclosure, the BTN3A activating antibody is a humanized antibody. Typically, a non-human antibody is humanized to reduce immunogenicity to humans, while having at least the same affinity (or superior affinity) of the parental non-human antibody. More particularly the BTN3A activating antibody is a humanized form of the antibodies 20.1, or 7.2 disclosed in WO2012080351. In specific embodiments, a humanized antibody comprises all 6 CDRs, of non-human antibody, e.g., the of the murine mAb 20.1 or 7.2, and FRs (or portions thereof) derived from the murine antibody sequences with one or more mutations to reduce immunogenicity. In other specific embodiments, it is a monovalent humanized BTN3A activating antibody such as a humanized Fab fragment having all 6 CDRs of mAb 103.2.

A humanized antibody optionally will also comprise at least a portion of a human constant region. Preferably, the recombinant antibody according to the disclosure is a humanized silent antibody, typically a humanized silent IgG1 or IgG4 antibody.

As used herein, the term “silent” antibody refers to an antibody that exhibits no or low FcγR binding and/or C1q binding as measured in binding assays such as those described in WO2020/025703. In one embodiment, the term “no or low FcγR and/or C1q binding” means that the silent antibody exhibits an FcγR and/or C1q binding that is at least below 50%, for example below 80% of the FcγR and/or C1q binding that is observed with the corresponding antibody with wild type human IgG1 or IgG4 isotype.

Examples BTN3A activating antibodies are described in the paragraphs below. In some embodiments, the BTN3A activating antibody is selected from the group consisting of BTN3A activating antibodies such as those described in the International Patent Applications WO2012/080769; WO2012/080351, and WO2020/025703 and WO2020/136218.

In some particular embodiments, BTN3A activating antibody is selected from the humanized antibodies described in WO2020025703 or is a humanized version of the BTN3A activating antibodies described in WO2012/080769, WO2012/080351 and WO2020/136218.

In some embodiments, the BTN3A antibody can be selected from mAb 20.1, and mAb 7.2, which are obtainable from one of the hybridomas accessible under CNCM deposit number I-4401, and I-4402 such as described in WO2012080769 and WO2012080351 or humanized versions thereof.

In some embodiments, the BTN3A activating antibody comprises the six CDRs (CDR1 (also called HCDR1), VH CDR2 (also called HCDR2), VH CDR3 (also called HCDR1), VL CDR1 (also called LCDR1), VL CDR2s (also called LCDR2), VL CDR3s (also called HCDR3)) of the antibody 20.1, or 7.2 described in WO2012080769 and WO2012080351 or any of mAbs 1-5 as described in WO2020025703, monovalent fragment of mAb103.2 as described in WO2020/136218.

In particular embodiments, the BTN3A activating antibody comprises HCDR1, HCDR2, HCDR3, LCDR1, LCDR2, and HCDR3 as shown in Table 1 below:

TABLE 1
CDR regions of mAb 20.1, mAb 7.2, humanized
mAb 103.2, or mAb1-mAb 6 as defined in WO2020/025703,
according to Kabat numbering.

| Original | | | | | | |
|---|---|---|---|---|---|---|
| antibody | HCDR1 | HCDR2 | HCDR3 | LCDR1 | LCDR2 | LCDR3 |
| mAb 20.1 | SEQ ID | SEQ ID | SEQ ID | SEQ ID | SEQ ID | SEQ ID |
| mAb 3, mAb 6 | NO: 5 | NO: 6 | NO: 7 | NO: 8 | NO: 9 | NO: 10 |
| mAb 7.2 | SEQ ID | SEQ ID | SEQ ID | SEQ ID | SEQ ID | SEQ ID |
| mAb1 | NO: 11 | NO: 12 | NO: 13 | NO: 14 | NO: 15 | NO: 16 |
| mAb2 | | | | | | |
| mAb4 | | | | | | |
| mAb5 | | | | | | |
| Humanized | SEQ ID | SEQ ID | SEQ ID | SEQ ID | SEQ ID | SEQ ID |
| mAb 103.2 | NO: 17 | NO: 18 | NO: 19 | NO: 20 | NO: 21 | NO: 22 |

In some embodiments, the antibodies for use as disclosed herein comprises 6 CDR regions which are respectively 100% identical to the 6 CDR regions of mAb 20.1, mAb 7.2 or humanized mAb103.2 as described in Table 1, notably of mAb 20.1.

Other antibodies as disclosed herein include those having amino acids that have been mutated by amino acid deletion, insertion or substitution, yet have at least 60, 70, 80, 90, 95, 96, 97, 98, 99 or 100 percent identity in the CDR regions as compared to the 6 CDR regions of the antibodies 20.1, or 7.2 described in WO2012080769 and WO2012080351 or of humanized mAb 103.2 described in WO2020/136218, notably as compared to the 6 CDR regions defined in Table 1.

BTN3A activating antibodies for use of the present disclosure include also those having at least 90%, notably at least, 95, 96, 97, 98, 99 or 100% identity with the VH and VL regions as defined in Table 2. More particularly, BTN3A activating antibodies for use of the disclosure include the selected humanized recombinant antibodies mAb1, mAb2, mAb3, mAb4, mAb5 and mAb6, which are structurally characterized by their variable heavy and light chain amino acid sequences and human constant regions (isotypes) as described in the Table 2 below:

TABLE 2
Variable heavy and light chain amino
acid sequences of mAb1-mAb6

| | VH | VL | |
|---|---|---|---|
| | Amino acid | Amino acid | Isotype constant |
| Antibody | sequence | sequence | region |
| mAb1 | SEQ ID NO: 3 | SEQ ID NO: 4 | Silent IgG1 |
| | (VH2 7.2) | (Vk1 7.2) | L247F/L248E/P350S |
| mAb2 | SEQ ID NO: 3 | SEQ ID NO: 25 | Silent IgG1 |
| | (VH2 7.2) | (Vk2 7.2) | L247F/L248E/P350S |
| mAb3 | SEQ ID NO: 1 | SEQ ID NO: 2 | Silent IgG1 |
| | | | L247F/L248E/P350S |
| mAb4 | SEQ ID NO: 3 | SEQ ID NO: 4 | IgG4 S241P/L248E |
| | (VH2 7.2) | (Vk1 7.2) | |
| mAb5 | SEQ ID NO: 3 | SEQ ID NO: 25 | IgG4 S241P/L248E |
| | (VH2 7.2) | (Vk2 7.2) | |
| mAb6 | SEQ ID NO: 1 | SEQ ID NO: 2 | IgG4 S241P/L248E |

The corresponding amino acid and nucleotide coding sequence of the constant isotype regions of IgG1, IgG4 and their mutant versions IgG1 L247F/L248E/P350S and IgG4 S241P/L248E used for generating mAb1 to mAb6 are well-known in the art (Oganesyan et al., 2008; Reddy et al., 2000).

The C-terminal lysine found in IgG may be naturally cleaved off and this modification does not affect the properties of the antibody; so, this residue may additionally be deleted in the constructs of mAb1 to mAb6.

Full length light and heavy chains and coding sequences for making preferred humanized antibodies for use of the present disclosure, mAb1 and mAb3, are shown in the Table 3 below.

TABLE 3
DNA coding sequences for mAb1 and mAb3

| Antibody | Amino acid sequence | DNA coding sequences |
|---|---|---|
| mAb1 | Heavy Chain: SEQ ID NO: 26 | Heavy Chain: SEQ ID NO: 30 |
| | Light Chain: SEQ ID NO: 27 | Light Chain: SEQ ID NO: 31 |
| mAb 3 | Heavy Chain: SEQ ID NO: 23 | VH: SEQ ID NO: 28 |
| | Light Chain: SEQ ID NO: 24 | VL: SEQ ID NO: 29 |

### Functional Variants of mAb 20.1, mAb 7.2 or BTN3A Activating Fragments of mAb 103.2, for Use According to the Present Disclosure

Analysis of epitope mapping indicates that the reference mAb 20.1 binds residues on positions: 79, 83 and 88 of the human BTN3A1 of SEQ ID NO:32. Thus the present disclosure encompasses use of BTN3A antibodies that bind an epitope comprising amino acid residues located in positions 79 to 88 of SEQ ID N°32, and that have one or more of the functional properties as previously defined and as further reminded below, in particular that have one or more of the functional properties of the reference mAb 20.1 or its humanized form mAb 3.

As also indicated by epitope mapping analysis, the reference mAb 7.2 binds residues positions: 73, 79, 83, 88, 90, 93 of the human BTN3A1 of SEQ ID NO:32. Thus the present disclosure encompasses BTN3A antibodies that bind an epitope comprising amino acid residues located in positions 73 to 93 of SEQ ID N°32, and most particularly, an epitope comprising amino acid residues on positions: 73, 79, 83, 88, 90 and 93 of SEQ ID N°32 and that have one or more of the functional properties as previously defined and as further reminded below, in particular that have one or more of the functional properties of the reference mAb 7.2 or its humanized form mAb 1.

In yet other embodiments, a functional variant antibody of the disclosure has full length heavy and light chain amino acid sequences; or variable region heavy and light chain amino acid sequences, or all 6 CDR regions amino acid sequences that are homologous or more specifically identical to the corresponding amino acid sequences of any one of the reference antibody mAb 20.1 or mAb 7.2 or their humanized forms (mAb 3 or mAb 1 respectively), described above, and wherein such functional variant antibodies retain the desired functional properties of said reference antibody.

In yet other embodiments, a functional variant antibody of the disclosure is a BTN3A activating fragment of mAb 103.2, such as Fab fragment having variable region heavy and light chain amino acid sequences, or all 6 CDR regions amino acid sequences that are homologous or more specifically identical to the corresponding amino acid sequences of any the reference antibody mAb 103.2 or a humanized form thereof, described above, and wherein such functional variant antibodies retain the desired functional properties of said reference antibody.

A functional variant of the reference mAb 20.1 antibody, or its humanized form mAb 3, or of the reference antibody mAb 7.2 or its humanized form mAb 1, or of the reference activating fragment of mAb 103.2 or its humanized form, notably a functional variant having VH and VL, or all 6 CDRs used in the context of a monoclonal antibody of the present disclosure still allows the antibody to retain at least a substantial proportion (at least about 50%, 60%, 70%, 80%, 90%, 95% or 100%) of the affinity (typically assessed by K_{D} as measured by surface plasmon resonance (SPR)) of the parent antibody (e.g.: mAb 3 or mAb 1) and in some cases such a functional variant may be associated with greater affinity, selectivity and/or specificity than the reference antibody (e.g.: mAb 3 or mAb 1 or Fab fragment of mAb 103.2).

Desired functional properties of the reference antibody, typically when the reference antibody is mAb 3 or mAb 1 or Fab fragment of mAb 103.2, or of any example reference antibody as herein disclosed, may be selected from the group consisting of:
(i) specificity for BTN3A1, in particular the property of binding to human BTN3A1 as measured by surface plasmon resonance (SPR) assay; for example as described in the Examples;
(ii) in vitro induction of the activation of γδ T cells, typically Vγ9Vδ2 T cells, in co-culture with BTN3A expressing cells, as measured in a degranulation assay, for example as described in the Examples;
(iii) reduction of C. burnetii bacterial load of monocytes in the presence of Vγ9Vδ2 T cells;
(iv) in vitro increase of cytotoxic activity of Vγ9Vδ2 T cells towards C. burnetii infected monocytes after 4 hours of co-culture,
(v) in vitro increase of Vγ9Vδ2 T cells degranulation towards peripheral blood mononuclear cells from C. burnetii infected patients; and/or,
(vi) in vitro increases of cytotoxic activity of Vγ9Vδ2 T cells towards SARS-Cov2 infected cells, for example as measured in vitro in co-cultures of infected cells with Vγ9Vδ2 T cells.

Typically, functional properties according to points (i) to (vi) above of a functional variant of the reference mAb 3 or 1 or activating fragment of mAb 103.2 are substantially equal or superior to the corresponding functional properties of the corresponding reference antibody mAb 3 or mAb 1 or activating fragment of mAb 103.2 as described above. By substantially equal it is herein intended that the functional variant retains at least about 50%, 60%, 70%, 80%, 90%, 95%, 96%, 97%, 98%, 99% or 100% of the corresponding functional property of the reference mAb 3 or mAb 1 or activating fragment of mAb 103.2.

In specific embodiments, the BTN3A activating antibodies for use according to the present disclosure are functional variants of mAb 20.1 or its humanized form of mAb 3, or mAb 7.2 or its humanized form of mAb 1, or activating fragment of mAb 103.2 or its humanized form, having not more than 1, 2, 3 or 4 amino acid variations (including deletion, insertion, or substitution) in one or more CDRs, as compared to the CDR sequences of the antibodies 20.1, or 7.2 or activating fragment of mAb 103.2 respectively or, more particularly as compared to the CDR sequences of mAb 20.1.

For example, the present disclosure relates to functional variant antibodies of the reference mAb 20.1 or its humanized form mAb 3, comprising a variable heavy chain (VH) and a variable light chain (VL) sequences where the CDR sequences, i.e., the 6 CDR regions; HCDR1, HCDR2, HCDR3, LCDR1, LCDR2, LCDR3 share at least 60, 70, 90, 95 or 100 percent sequence identity to the corresponding CDR sequences of mAb 20.1 or its humanized form mAb 3 reference antibody, as defined in SEQ ID NO:5-10, wherein said functional variant antibody specifically binds to BTN3A, and the antibody exhibits at least one of the following functional properties i) to iii):
(i) specificity for BTN3A1, in particular the property of binding to human BTN3A1 with a K_{D} of 10 nM or less, preferably with a K_{D} of 5 nM or less, or with a K_{D} of 5 nM or less, as measured by surface plasmon resonance (SPR) for example as described in the Examples;
(ii) binding to human PBMCs with an EC₅₀ of 50 μg/ml or below, preferably of 10 μg/ml or below, as measured in a flow cytometry assay, for example as described in the Examples;
(iii) induction of the activation of γδ T cells, typically Vγ9Vδ2 T cells, in co-culture with BTN3A expressing cells, with an EC₅₀ below 5 μg/ml, preferably of 1 μg/ml or below, as measured in a degranulation assay, for example as described in the Examples.

Most preferably it exhibits properties i)-iii).

The present disclosure also relates to functional variant antibodies of the reference mAb 7.2 or its humanized form mAb 1, comprising a variable heavy chain (V_{H}) and a variable light chain (V_{L}) sequences where the CDR sequences, i.e., the 6 CDR regions; HCDR1, HCDR2, HCDR3, LCDR1, LCDR2, LCDR3 share at least 60, 70, 90, 95 or 100 percent sequence identity to the corresponding CDR sequences of mAb 7.2 or the mAb 1 reference antibody, as defined in SEQ ID NO: 11-16, wherein said functional variant antibody specifically binds to BTN3A1, and the antibody exhibits at least one of the following functional properties:
(i) specificity for BTN3A1, in particular the property of binding to human BTN3A1 with a K_{D} of 10 nM or less, preferably with a K_{D} of 5 nM or less, or with a K_{D} of 5 nM or less, as measured by surface plasmon resonance (SPR) for example as described in the Examples;
(ii) binding to human PBMCs with an EC₅₀ of 50 μg/ml or below, preferably of 10 μg/ml or below, as measured in a flow cytometry assay, for example as described in the Examples;
(iii) induction of the activation of γδ T cells, typically Vγ9Vδ2 T cells, in co-culture with BTN3A expressing cells, with an EC50 below 5 μg/ml, preferably of 1 μg/ml or below, as measured in a degranulation assay, for example as described in the Examples.

Preferably said functional variants exhibit all functional activities i) to iii).

The present disclosure also relates to functional variant antibodies of the reference Fab fragment mAb 103.2 or its humanized form, comprising a variable heavy chain (V_{H}) and a variable light chain (V_{L}) sequences where the CDR sequences, i.e., the 6 CDR regions; HCDR1, HCDR2, HCDR3, LCDR1, LCDR2, LCDR3 share at least 60, 70, 90, 95 or 100 percent sequence identity to the corresponding CDR sequences of reference Fab fragment of mAb 103.2 antibody, as defined in SEQ ID NO: 17-22, wherein said functional variant antibody specifically binds to BTN3A1, and the antibody exhibits at least one of the following functional properties:
(i) specificity for BTN3A1, in particular the property of binding to human BTN3A1 with a K_{D} of 10 nM or less, preferably with a K_{D} of 5 nM or less, or with a K_{D} of 5 nM or less, as measured by surface plasmon resonance (SPR) for example as described in the Examples;
(ii) binding to human PBMCs with an EC₅₀ of 50 μg/ml or below, preferably of 10 μg/ml or below, as measured in a flow cytometry assay, for example as described in the Examples;
(iii) induction of the activation of γδ T cells, typically Vγ9Vδ2 T cells, in co-culture with BTN3A expressing cells, with an EC50 below 5 μg/ml, preferably of 1 μg/ml or below, as measured in a degranulation assay, for example as described in the Examples.

Preferably said functional variants exhibit all functional activities i) to iii).

It further relates to functional variant antibodies of the mAb 3 reference antibody comprising a heavy chain variable region and a light chain variable region that are at least 80%, 90%, or at least 95, 96%, 97%, 98%, 99% or 100% identical to the corresponding heavy and light chain variable regions of said mAb 3 reference antibody, as defined respectively in SEQ ID NO: 1 and 2; the functional variant antibody specifically binds to BTN3A, and exhibits at least one of the following functional properties:
(i) specificity for BTN3A1, in particular the property of binding to human BTN3A1 with a K_{D} of 10 nM or less, preferably with a K_{D} of 5 nM or less, or with a K_{D} of 5 nM or less, as measured by surface plasmon resonance (SPR) for example as described in the Examples;
(ii) binding to human PBMCs with an EC₅₀ of 50 μg/ml or below, preferably of 10 μg/ml or below, as measured in a flow cytometry assay, for example as described in the Examples;
(iii) induction of the activation of γδ T cells, typically Vγ9Vδ2 T cells, in co-culture with BTN3A expressing cells, with an EC50 below 5 μg/ml, preferably of 1 μg/ml or below, as measured in a degranulation assay, for example as described in the Examples.

Preferably it exhibits properties i)-iii).

It further relates to functional variant antibodies of the mAb 1 reference antibody comprising a heavy chain variable region and a light chain variable region that are at least 80%, 90%, or at least 95, 96%, 97%, 98%, 99% or 100% identical to the corresponding heavy and light chain variable regions of said mAb 1 reference antibody, as defined respectively in SEQ ID NO: 3 and 4; the functional variant antibody specifically binds to BTN3A, and exhibits at least one of the following functional properties:
(i) specificity for BTN3A1, in particular the property of binding to human BTN3A1 with a K_{D} of 10 nM or less, preferably with a K_{D} of 5 nM or less, or with a K_{D} of 5 nM or less, as measured by surface plasmon resonance (SPR) for example as described in the Examples;
(ii) binding to human PBMCs with an EC₅₀ of 50 μg/ml or below, preferably of 10 μg/ml or below, as measured in a flow cytometry assay, for example as described in the Examples;
(iii) induction of the activation of γδ T cells, typically Vγ9Vδ2 T cells, in co-culture with BTN3A expressing cells, with an EC50 below 5 μg/ml, preferably of 1 μg/ml or below, as measured in a degranulation assay, for example as described in the Examples.

In some embodiments, said functional variants exhibit all functional activities i) to iii).

It further relates to functional variant antibodies of the reference Fab fragment of mAb 103.2 comprising a heavy chain variable region and a light chain variable region that are at least 80%, 90%, or at least 95, 96%, 97%, 98%, 99% or 100% identical to the corresponding heavy and light chain variable regions of said Fab fragment of mAb 103.2, as defined respectively in SEQ ID NO: 63 and 64; the functional variant antibody specifically binds to BTN3A, and exhibits at least one of the following functional properties:
(i) specificity for BTN3A1, in particular the property of binding to human BTN3A1 with a K_{D} of 10 nM or less, preferably with a K_{D} of 5 nM or less, or with a K_{D} of 5 nM or less, as measured by surface plasmon resonance (SPR) for example as described in the Examples;
(ii) binding to human PBMCs with an EC₅₀ of 50 μg/ml or below, preferably of 10 μg/ml or below, as measured in a flow cytometry assay, for example as described in the Examples;
(iii) induction of the activation of γδ T cells, typically Vγ9Vδ2 T cells, in co-culture with BTN3A expressing cells, with an EC50 below 5 μg/ml, preferably of 1 μg/ml or below, as measured in a degranulation assay, for example as described in the Examples.

In some embodiments, said functional variants exhibit all functional activities i) to iii).

Typically, functional properties according to points (i) to (iii) above of a functional variant of the reference mAb 3 or mAb 1 or Fab fragment of mAb 103.2 are substantially equal or superior to the corresponding functional properties of the corresponding reference antibody mAb 3 or mAb 1 or Fab fragment of mAb 103.2 as described above. By substantially equal it is herein intended that the functional variant retains at least about 50%, 60%, 70%, 80%, 90%, 95%, 96%, 97%, 98%, 99% or 100% of the corresponding functional property of the reference mAb 3 or mAb 1 or Fab fragment of mAb 103.2.

The sequences of CDR variants may differ from the sequence of the CDRs of the parent antibody sequences through mostly conservative substitutions; for instance, at least 10, such as at least 9, 8, 7, 6, 5, 4, 3, 2 or 1 of the substitutions in the variant are conservative amino acid residue replacements.

Functional variant antibodies with mutant amino acid sequences can be obtained by mutagenesis (e.g., site-directed or PCR-mediated mutagenesis) of the coding nucleic acid molecules, followed by testing of the encoded altered antibody for retained function (i. e., the functions set forth above) using the functional assays described herein.

Antibodies that Cross-Compete the Reference mAb 3 or mAb 1 or Fab Fragment of mAb 103.2.

Additional antibodies with similar advantageous properties of the reference mAb 3 or the reference mAb 1 or Fab fragment of mAb 103.2 as disclosed herein can be identified based on their ability to cross-compete with (e.g., to competitively inhibit the binding of), in a statistically significant manner, said reference mAb 3 or mAb 1 or Fab fragment of mAb 103.2 as described above, in standard BTN3A1 binding assays.

Test antibodies may first be screened for their binding affinity to BTN3A1, for example from human recombinant antibody libraries using for example phage display technologies or from transgenic mouse expressing human variable region antibodies immunized with BTN3A1 antigens as assessed typically in the Examples (see Material and Methods section).

In another embodiment, antibodies for use according to the present disclosure include antibodies that bind to the same epitope as do at least the reference mAb 3 or the reference mAb 1 or Fab fragment of mAb 103.2 as described above.

The ability of a test antibody to cross-compete with, or inhibit the binding of antibodies of the present disclosure to human BTN3A1, demonstrates that the test antibody can compete with that antibody for binding to human BTN3A1; such an antibody may, according to non-limiting theory, bind to the same or a related (e.g., a structurally similar or spatially proximal) epitope on human BTN3A1 as the antibody with which it competes.

For example, the following test can be used to screen a BTN3A1 antibody for its ability to cross-compete with mAb 3 reference antibody and/or to screen a anti-BTN3A1 antibody for its ability to binds to the same epitope as said reference antibody: BTN3KO cells transfected with human BTN3A1 (typically HEK293T) can be stained with saturing concentration (e.g., 10 μg/mL) of the reference antibody mAb 3. Different doses of a test BTN3A1 mAbs can then be tested for their competitive potential with the mAb 3 reference antibody. The mAbs that do compete with the reference antibody will not be able to recognize BTN3A1 in the presence of such reference antibody. The data can be expressed as mean fluorescence intensity. Alternatively, competition assay can be performed in a binning assay as described in the Example section. Typically, binning experiment can be performed by immobilizing recombinant human BTN3A1 on a Biosensor and by presenting the reference antibody followed by the competing antibody.

The selected antibodies can be further tested and selected for the advantageous BTN3A activating properties as compared to mAb 3 or mAb 1 or Fab fragment of mAb 103.2 in particular as previously detailed.

In some embodiments, the antibodies for use of the present disclosure compete for binding to BTN3A antibodies described above, in particular an antibody for use of the present disclosure competes for binding with an antibody selected from mAb 20.1, and mAb 7.2, which are obtainable from one of the hybridomas accessible under CNCM deposit number I-4401, and I-4402 such as described in WO2012/080769 and WO2012/080351, as well as from mAbs 1-6 described in WO2020025703. In more particular embodiments, the antibodies for use of the present disclosure compete for binding with an antibody selected from mAb 20.1 as produced by the hybridomas deposited at the CNCM under deposit number I-4401, and an antibody having a heavy chain of SEQ ID NO:23 and a light chain of SEQ ID NO:24.

Accordingly, in one embodiment, the present disclosure provides an isolated antibody for use in treating infectious disorders as disclosed herein, wherein said isolated antibody competes with the reference mAb 3 or the reference mAb 1 or Fab fragment of mAb 103.2, from binding to BTN3A1, and wherein said antibody has one or more of the following properties:
(i) specificity for BTN3A1, in particular the property of binding to human BTN3A1 as measured by surface plasmon resonance (SPR) assay; for example as described in the Examples;
(ii) in vitro induction of the activation of γδ T cells, typically Vγ9Vδ2 T cells, in co-culture with BTN3A expressing cells, as measured in a degranulation assay, for example as described in the Examples;
(iii) reduction of C. burnetii bacterial load of monocytes in the presence of Vγ9Vδ2 T cells;
(iv) in vitro increase of cytotoxic activity of Vγ9Vδ2 T cells towards C. burnetii infected monocytes after 4 hours of co-culture,
(v) in vitro increase of Vγ9Vδ2 T cells degranulation towards peripheral blood mononuclear cells from C. burnetii infected patients; and/or,
(vi) in vitro increase of cytotoxic activity of Vγ9Vδ2 T cells towards SARS-Cov2 infected cells, for example as measured in vitro in co-cultures of infected cells with Vγ9Vδ2 T cells.

In specific embodiments, functional properties according to points (i) to (vi) above of an antibody that competes for binding to BTN3A1 with the reference mAb 3 or mAb 1 or Fab fragment of mAb 103.2 are substantially equal or superior to the corresponding functional properties of the reference antibody mAb 1 or mAb 3 or Fab fragment of mAb 103.2 respectively, as described above. By substantially equal it is herein intended that the functional variant retains at least about 50%, 60%, 70%, 80%, 90%, 95% or 100% of the corresponding functional property of the reference mAb 1 or mAb 3 or Fab fragment of mAb 103.2.

In a certain embodiment, the cross-blocking antibodies or antibody that competes for binding to BTN3A1 with the reference mAb 1 or mAb 3 or Fab fragment of mAb 103.2, is a chimeric, humanized or human recombinant antibody.

### Framework or Fc Engineering

The BTN3A activating antibodies for use of the disclosure can include modifications made to framework residues within VH and VL, to decrease its immunogenicity.

In some specific embodiments, the antibody for use of the disclosure is a humanized monoclonal antibody of the parent murine antibody mAb 20.1, including at least the following amino acid mutations in the VH framework regions (as compared to VH parental framework regions): V5Q; V11L; K12V; V20L; R66K; M69L; T75S; M801; E81Q; R83T; T87S; L108A; and at least the following amino acid mutations in the VK framework regions (as compared to VK framework regions): T5N; V15L; R18T; V191; K39R; K42N; A431; D70G; F73L; V104L.

In addition to modifications made within the framework regions, the antibodies of the disclosure may be engineered to include modifications within the Fc region, typically to alter one or more functional properties of the antibody, such as serum half-life, complement fixation, Fc receptor binding, and/or antigen-dependent cellular cytotoxicity.

Furthermore, an antibody for use of the disclosure may be chemically modified (e.g., one or more chemical moieties can be attached to the antibody) or be modified to alter its glycosylation, again to alter one or more functional properties of the antibody. Each of these embodiments is described in further detail below.

As used herein, the term “isotype constant region” or “Fc region” is used interchangeably to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody wherein the numbering is according to the EU numbering system. The C-terminal lysine (residue K447) of the Fc region may be removed, for example, during production or purification of the antibody or its corresponding codon deleted in the recombinant constructs. Accordingly, a composition of antibodies of the disclosure may comprise antibody populations with all K447 residues removed, antibody populations with no K447 residues removed, and antibody populations having a mixture of antibodies with and without the K447 residue.

In other embodiments, the Fc region is modified to decrease the ability of the antibody to mediate antibody dependent cellular cytotoxicity (ADCC) and/or to decrease the affinity of the antibody for an Fcγ receptor by modifying one or more amino acids. Such antibodies with decreased effector functions, and in particular decreased ADCC include silent antibodies.

In certain embodiments, the Fc domain of the IgG1 isotype is used. In some specific embodiments, a mutant variant of the IgG1 Fc fragment is used, e.g. a silent IgG1 Fc which reduces or eliminates the ability of the fusion polypeptide to mediate antibody dependent cellular cytotoxicity (ADCC) and/or to bind to an Fcγ receptor.

In certain embodiments, the Fc domain of the IgG4 isotype is used. In some specific embodiments, a mutant variant of the IgG4 Fc fragment is used, e.g. a silent IgG4 Fc which reduces or eliminates the ability of the fusion polypeptide to mediate antibody dependent cellular cytotoxicity (ADCC) and/or to bind to an Fcγ receptor.

Silenced effector functions can be obtained by mutation in the Fc constant part of the antibodies and have been described in the Art (Baudino et al., 2008; Strohl, 2009). Examples of silent IgG1 antibodies comprise the triple mutant variant IgG1 L247F L248E P350S. Examples of silent IgG4 antibodies comprise the double mutant variant IgG4 S241P L248E.

In certain embodiments, the Fc domain is a silent Fc mutant preventing glycosylation at position 314 of the Fc domain. For example, the Fc domain contains an amino acid substitution of asparagine at position 314. An example of such amino acid substitution is the replacement of N314 by a glycine or an alanine.

In still other embodiments, the glycosylation of an antibody is modified. For example, an aglycoslated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Another modification of the antibodies herein that is contemplated for use according to the present disclosure is pegylation or hesylation or related technologies. An antibody can be pegylated to, for example, increase the biological (e.g., serum) half-life of the antibody. To pegylate an antibody, the antibody, or fragment thereof, typically is reacting with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the disclosure. See for example, EP 0 154 316 by Nishimura et al. and EP 0 401 384 by Ishikawa et al.

In certain embodiments, the C-terminal lysine commonly present on human IgG heavy chain constant domains, is engineered out to reduce heterogeneity due to the cleavage of this reduce commonly observed during manufacturing or storage. Such modifications do not perceptible change the desirable functions of these antibodies, while conferring the benefit of stability to these molecules.

### Nucleic Acid Molecules Encoding Antibodies of the Disclosure

Also disclosed herein are the nucleic acid molecules that encode the BTN3A activating antibodies for use according to the present disclosure. Examples of variable light chain and heavy chain nucleotide sequences are those encoding the variable light chain and heavy chain amino acid sequences of any one of the above disclosed exemplary BTN3A activating antibodies, in particular mAb 7.2, mAb 20.1 and their humanized forms, such as mAb1, mAb2, mAb3, mAb4, mAb5, and mAb 6, some of them being easily derived from the Table 1 and Table 2, and using the genetic code and, optionally taking into account the codon bias depending on the host cell species.

The present disclosure also pertains to nucleic acid molecules that derive from the latter sequences having been optimized for protein expression in mammalian cells, for example, CHO cell lines.

Further disclosed herein are the nucleic acid molecules encoding respectively a heavy chain of humanized form of mAb 20.1 of SEQ ID NO:23 and a light chain of humanized form of mAb 20.1 of SEQ ID NO:24.

In specific embodiments, the antibodies for use of the present disclosure are humanized form of mAb 20.1 having a VH as encoded by SEQ ID NO:28 and a VL as encoded by SEQ ID NO: 29.

Further disclosed herein are the nucleic acid molecules of SEQ ID NO:28 and SEQ ID NO:29 encoding respectively a VH and a VL of humanized form of mAb 20.1.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art (Ausubel et al., 1988). A nucleic acid of the disclosure can be, for example, DNA or RNA and may or may not contain intronic sequences. In an embodiment, the nucleic acid may be present in a vector such as a phage display vector, or in a recombinant plasmid vector.

Nucleic acids of the disclosure can be obtained using standard molecular biology techniques. Once DNA fragments encoding, for example, VH and VL segments are obtained, these DNA fragments can be further manipulated by standard recombinant DNA techniques, for example to convert the variable region genes to full-length antibody chain genes, to Fab fragment genes or to an scFv gene. In these manipulations, a VL- or VH-encoding DNA fragment (for example VL and VH as defined in Table 2) is operatively linked to another DNA molecule, or to a fragment encoding another protein, such as an antibody constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (Kabat et al., 1992) and DNA fragments encompassing these regions can be obtained by standard PCR amplification. The heavy chain constant region can be an IgG1, IgG2, IgG3, IgG4, IgA, IgE, IgM or IgD constant region. In some embodiments, the heavy chain constant region is selected among IgG1 isotypes, for example human IgG1 isotype. In other embodiments, the heavy chain constant region is selected among IgG4 isotypes, for example human IgG4 isotype. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as to a Fab light chain gene) by operatively linking the VL-encoding DNA to another DNA molecule encoding the light chain constant region, CL. The sequences of human light chain constant region genes are known in the art (Kabat et al., 1992) and DNA fragments encompassing these regions can be obtained by standard PCR amplification. The light chain constant region can be a kappa or a lambda constant region.

To create an scFv gene, the VH- and VL-encoding DNA fragments are operatively linked to another fragment encoding a flexible linker, e.g., encoding the amino acid sequence (Gly4-Ser) 3, such that the VH and VL sequences can be expressed as a contiguous single-chain protein, with the VL and VH regions joined by the flexible linker (Bird et al., 1988; Huston et al., 1988; McCafferty et al., 1990).

### Methods for Producing Recombinant Antibodies for Use According to the Present Disclosure

Antibodies of the present disclosure can be produced in a host cell transfectoma using, for example, a combination of recombinant DNA techniques and gene transfection methods as is well known in the art (Morrison, 1985).

For example, to express the antibodies, or antibody fragments thereof, DNAs encoding partial or full-length light and heavy chains can be obtained by standard molecular biology or biochemistry techniques (e.g., DNA chemical synthesis, PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the VH segment is operatively linked to the CH segment(s) within the vector and the VL segment is operatively linked to the CL segment within the vector. Additionally, or alternatively, the recombinant expression vector can encode a signal peptide that facilitates secretion of the antibody chain from a host cell. The antibody chain gene can be cloned into the vector such that the signal peptide is linked in frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors disclosed herein carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel's publication (Goeddel, 1990). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1 (Takebe et al., 1988).

In addition to the antibody chain genes and regulatory sequences, the recombinant expression vectors of the present disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5, 179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies of the present disclosure in either prokaryotic or eukaryotic host cells. Expression of antibodies in eukaryotic cells, for example mammalian host cells, yeast or filamentous fungi, is discussed because such eukaryotic cells, and in particular mammalian cells, are more likely than prokaryotic cells to assemble and secrete a properly folded and immunologically active antibody.

In one specific embodiment, a cloning or expression vector according to the disclosure comprises one of the coding sequences of the heavy and light chains of any one of mAb1, or mAb3 operatively linked to suitable promoter sequences.

Mammalian host cells for expressing the recombinant antibodies of the disclosure include Chinese Hamster Ovary (CHO cells) including dhfr-CHO cells (described in Urlaub and Chasin, 1980) used with a DHFR selectable marker (as described in Kaufman and Sharp, 1982), CHOK1 dhfr+ cell lines, NSO myeloma cells, COS cells and SP2 cells, for example GS CHO cell lines together with GS Xceed™ gene expression system (Lonza). When recombinant expression vectors encoding antibody genes are introduced into mammalian host cells, the antibodies are produced by culturing the host cells for a period of time sufficient for expression of the antibody in the host cells and, optionally, secretion of the antibody into the culture medium in which the host cells are grown. Antibodies can be recovered and purified for example from the culture medium after their secretion using standard protein purification methods (Shukla et al., 2007).

In one specific embodiment, the host cell of the disclosure is a host cell transfected with an expression vector having the coding sequences suitable for the expression of mAb1 or mAb3 or Fab fragment of mAb 103.2 respectively, operatively linked to suitable promoter sequences.

For example, the present disclosure relates to a host cell comprising at least the nucleic acids of SEQ ID NO:28 and 29 encoding respectively the VH and VL of mAb3.

The latter host cells may then be further cultured under suitable conditions for the expression and production of an antibody of the disclosure selected from the group consisting of mAb1, or mAb3, or Fab fragment of mAb 103.2, respectively.

Alternatively, cell free expression systems may be used for the production of any of mAb3, or mAb1. Typically, methods of cell-free expression of proteins or antibodies are already described (Stech et al., 2017).

### Pharmaceutical Compositions

In another aspect, the present disclosure provides a composition for use in treating infectious disorders as disclosed hereafter, e.g., a pharmaceutical composition, containing BTN3A activating antibodies, in particular, a BTN3A activating antibody selected from the group consisting of mAb 20.1, a BTN3A activating antibody having the 6 CDRs of mAb 20.1 of SEQ ID NO: 5-10, a BTN3A activating antibody having VH of SEQ ID NO: 1 and VL of SEQ ID NO: 2, a BTN3A activating antibody which is humanized form of mAb 20.1, and mAb3 having heavy chain of SEQ ID NO:23 and light chain of SEQ ID NO:24, and their antigen-binding portions, formulated together with a pharmaceutically acceptable carrier.

Such compositions may include one or a combination of (e.g., two or more different) BTN3A activating antibodies, as described above.

Pharmaceutical compositions disclosed herein also can also include additional active therapeutic agents. For example, the pharmaceutical compositions can include an anti-BTN3A antibody of the present disclosure, for example one antibody selected from the group consisting of the group consisting of mAb 20.1, a BTN3A activating antibody having the 6 CDRs of mAb 20.1 of SEQ ID NO: 5-10, a BTN3A activating antibody having VH of SEQ ID NO: 1 and VL of SEQ ID NO: 2, a BTN3A activating antibody which is humanized form of mAb 20.1, and mAb3 having heavy chain of SEQ ID NO:23 and light chain of SEQ ID NO:24, or their antigen-binding portions, combined with at least one anti-viral, anti-inflammatory or anti-bacterial agent. Examples of such other active therapeutic agents that can be used are described in greater detail below in the section on uses of the antibodies of the disclosure.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). In one embodiment, the carrier should be suitable for subcutaneous route or intravenous route. Depending on the route of administration, the active compound, i.e., the BTN3A activating antibody, may be coated in a material to protect the compound from the action of acids and other natural conditions that may inactivate the compound.

Sterile phosphate-buffered saline is one example of a pharmaceutically acceptable carrier. Other suitable carriers are well-known to those in the art. (Remington and Gennaro, 1995) Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc.

The pharmaceutical compositions for use according to the disclosure can be formulated for a topical, oral, parenteral, intranasal, intravenous, intramuscular, subcutaneous or intraocular administration and the like.

Preferably, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the BTN3A activating antibody may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders or lyophilisates for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

A BTN3A activating antibody for use according to the present disclosure can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The BTN3A activating antibodies for use according to the present disclosure may be formulated within a therapeutic mixture to comprise about 0.001 to 1 gram, or about 1 to 400 milligrams, or about 1 to 200 milligrams per dose. Multiple doses can also be administered.

Suitable formulation for solution for infusion or subcutaneous injection of antibodies have been described in the art and for example are reviewed in Cui et al (Drug Dev Ind Pharm 2017, 43 (4): 519-530).

### Preferred Embodiments of BTN3A Activating Antibodies for Use According to the Present Disclosure, their Pharmaceutical Compositions and Methods for Preparing the Same

#1. An isolated BTN3A activating antibody comprising: a variable heavy chain polypeptide VH of SEQ ID NO: 1 and a variable light chain polypeptide VL of SEQ ID NO:2
#2. An isolated BTN3A activating antibody comprising the 6 CDRs of SEQ ID NO:5-10, and a variable heavy chain polypeptide VH having at least 90% identity to SEQ ID NO:1 and a variable light chain polypeptide VL having at least 90% identity to SEQ ID NO:2
#3. The isolated BTN3A activating antibody of #1 or #2, which comprises a human IgG1 constant region, optionally wherein human IgG1 constant region is mutated or chemically modified such that said mutant or chemically modified IgG1 constant region confers no or decreased binding to Fcγ receptors when compared to a corresponding antibody with wild type human IgG1 isotype constant region.
#4. The isolated BTN3A activating antibody of any of #1 to #3, wherein said antibody exhibit one or more of the following properties:
   (i) it binds to human BTN3A1 with a K_{D} of 10 nM or less, preferably with a K_{D} of 5 nM or less, or with a K_{D} of 5 nM or less, as measured by surface plasmon resonance (SPR) for example as described in the Examples;
   (ii) it binds to human PBMCs with an EC₅₀ of 50 μg/ml or below, preferably of 10 μg/ml or below, as measured in a flow cytometry assay, for example as described in the Examples;
   (iii) it induces in vitro the activation of γδ T cells, typically Vγ9Vδ2 T cells, in co-culture with BTN3A expressing cells, with an EC50 below 5 μg/ml, preferably of 1 μg/ml or below, as measured in a degranulation assay, for example as described in the Examples.
#5. The isolated BTN3A activating antibody of any of #1 to #4, which comprises a heavy chain polypeptide of SEQ ID NO:23 and a light chain polypeptide of SEQ ID NO:24.
#6. A nucleic acid encoding the heavy and light chains of a BTN3A activating antibody of any of #1 to #5, for example comprising the nucleotide sequences of SEQ ID NO:28 and SEQ ID NO: 29.
#7. An expression vector for the recombinant production of a BTN3A activating antibody according to #1 in a host cell, comprising at least one nucleic acid encoding said BTN3A activating antibody.
#8. A host cell comprising an expression vector according to Claim #7.
#9. A pharmaceutical composition comprising an anti-BTN3A antibody as defined in any one of #1-#5, in combination with one or more of a pharmaceutically acceptable excipient, diluent or carrier, optionally comprising other active ingredients, for example a cytokine, such as IL-2 or IL-15, or their functional derivatives and pegylated versions.
#10. The pharmaceutical composition of Claim #9 which is a lyophilizate formulation, a solution in a pre-filled syringe or a solution in a vial.
#11. The isolated BTN3A activating antibody of any one of #1-#5 or a pharmaceutical composition of Claim #9 or #10, for use as a therapeutic.
#12. A process for the production of a BTN3A antibody of any one of #1-#5, comprising: (i) culturing the host cell of #8 for expression of said antibody by the host cell; optionally (ii) purifying said antibody; and (iii) recovering the antibody.

### Uses of the BTN3A Activating Antibodies in Treating Infectious Disorders

The inventors have found that BTN3A activating antibodies, such as mAb 20.1, have the capacity to
(i) potentiate in vitro the reduction of C. burnetii bacterial load of monocytes in the presence of Vγ9Vδ2 T cells
(ii) increase in vitro cytotoxic activity of Vγ9Vδ2 T cells towards C. burnetii infected monocytes as measured in vitro with co-cultures of infected monocytes, typically after 4 hours of co-culture, and/or

They have also found that BTN3A activating antibodies, such as mAb 20.1 have the capacity to potentiate the inhibition of SARS-Cov2 replication, for example as measured in vitro in co-cultures of infected cells with Vγ9Vδ2 T cells.

Accordingly, the present disclosure relates to BTN3A activating antibodies, in particular the specific BTN3A activating antibodies as described above, for use in treating infectious disorders, more specifically selected from viral or bacterial infectious disorders.

As used herein, the term “treat” “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease. In particular, with reference to the treatment of an infectious disorder, the term “treatment” may refer to the prevention of infection by the infectious agent, inhibition of the replication of the infectious agent, reduction of the severity of one or more of the symptoms associated to the infection, or eradication of the infectious agent.

In preferred embodiments, the subject is a human subject.

The BTN3A activating antibodies for use as disclosed above may be administered as the sole active ingredient or in conjunction with, e.g. as an adjuvant or in combination to other drugs e.g. cytokines, anti-viral, anti-inflammatory agents, for the treatment or prevention of diseases mentioned above.

For example, the antibodies for use as disclosed above may be used in combination with cytokines, anti-viral agents, anti-bacterial agents, or anti-inflammatory agents.

Examples of cytokines includes cytokines for expanding and/or activating Vγ9Vδ2T cells in vivo, including without limitation interleukin 2 (IL-2) (Choudhry H et al, 2018, Biomed Res Int. 2018 May 6), interleukin 15 (IL-15) (Patidar M et al., Cytokine Growth Factor Rev. 2016 October; 31:49-59), or their derivatives. The term derivative is used for any cytokine modifications that can rely on PEGylation (e.g. conjugation to polyethylene glycol (PEG) chains), mutation such as amino acid deletion, substitution or insertion, or association with potentiating agents (for example IL15/IL15Ra complexes fused to an IgG1 Fc, in which IL-15 is additionally mutated (asn72asp) that further increase biological activity making this complex an IL-2 and IL-15Rβγ superagonist (Rhode P R et al, Cancer Immunol Res. 2016; 4 (1): 49-60)) (Barroso-Sousa R et al, Curr Oncol Rep. 2018 Nov. 15; 21 (1): 1).

The term “IL-2” has its general meaning and refers to the human interleukin-2. IL-2 is part of the body's natural immune response. IL-2 mainly regulates lymphocyte activity by binding to IL-2 receptors.

The term “IL-15” has its general meaning and refers to the human interleukin-15. Like IL-2, IL-15 binds to and signals through a complex composed of IL-2/IL-15 receptor beta chain (CD122) and the common gamma chain (gamma-C, CD132). IL-15 regulates the activation and proliferation of T and natural killer (NK) cells.

A method of use as defined herein may therefore comprises co-administration, e.g. concomitantly or in sequence, of a therapeutically effective amount of a BTN3A activating antibody, and at least one second drug substance, said second drug substance being an anti-viral or anti-bacterial, anti-inflammatory or cytokines, e.g. IL-2 or IL-15, or a cell therapy product (such as γδ T cells), e.g. as indicated above.

### Uses of the BTN3A Activating Antibodies in Treating Disorder Caused by SARS Cov2 Infection

In specific embodiment, the present disclosure relates to a BTN3A activating antibodies, in particular the specific BTN3A activating antibodies as described above, for use in treating a disorder caused by SARS-Cov2 infection in a subject in need thereof, typically Covid-19.

In specific embodiment, the present disclosure relates to a method for treating a disorder caused by SARS-Cov2 infection in a subject in need thereof, typically Covid-19, said method comprising administering a therapeutically efficient amount of an anti-BTN3A activating antibody, in said subject.

In specific embodiment, the present disclosure relates to the use of a BTN3A activating antibody in the preparation of a medicament for treating a disorder caused by SARS-Cov2 infection, typically Covid-19, in a subject in need thereof.

In specific embodiment, the subject eligible for such treatment has been diagnosed as being infected by SARS-Cov2.

In specific embodiment, the subject is selected among the subject having mild or moderate COVID-19.

In specific embodiment, the subject is selected among the subject at high risk of progression to severe COVID-19. Risk factors include without limitation risk factors include (listed alphabetically) age (risk increases with each decade after age 50), cancer, cardiovascular disease, chronic kidney disease, chronic lung disease, diabetes, immunocompromising conditions or receipt of immunosuppressive medications, obesity (body mass index ≥30), pregnancy, and sickle cell disease.

In specific embodiments, anti-viral or anti-inflammatory drugs may be used in combination with BTN3A activating antibody for treating said disorder caused by SARS-Cov2. Examples of such anti-viral or anti-inflammatory drugs useful for treating SARS-Cov2 include without limitation remdesivir, baricitinib, bamlanivimab, bamlanivimab/etesevimab, casirivimab/imdevimab, dexamethasone, budesonide and tocilizumab.

Uses of the BTN3A Activating Antibodies in Treating Disorder Caused by Coxiella burnetii

In specific embodiment, the present disclosure relates to a BTN3A activating antibodies, in particular the specific BTN3A activating antibodies as described above, for use in treating a disorder caused by Coxiella burnetii infection in a subject in need thereof, typically Q fever.

In specific embodiment, the present disclosure relates to a method for treating a disorder caused by Coxiella burnetii infection in a subject in need thereof, typically Q fever, said method comprising administering a therapeutically efficient amount of a BTN3A activating antibody, in said subject.

In specific embodiment, the present disclosure relates to the use of a BTN3A activating antibody in the preparation of a medicament for treating a disorder caused by Coxiella burnetii infection, typically Q fever, in a subject in need thereof.

In specific embodiment, the subject eligible for such treatment has been diagnosed as being infected by Coxiella burnetii.

In specific embodiments, anti-bacterial (such as antibiotics) or anti-inflammatory drugs may be used in combination with BTN3A activating antibody for treating said disorder caused by Coxiella burnetii, including without limitation, doxycycline, tetracycline, chloramphenicol, ciprofloxacin, ofloxacin, and hydroxychloroquine.

Other specific embodiments for use of BTN3A activating antibodies are disclosed hereafter without limitation.

### Specific Embodiments for Use of BTN3A Activating Antibodies

#1. A BTN3A activating antibody, for use in treating infectious disorders in a human subject in need thereof.
#2. The BTN3A activating antibody for use according to #1, wherein said infectious disorder is a disorder caused by Coxiella burnetii infection, typically Q fever.
#3. The BTN3A activating antibody for use according to #1, wherein said infectious disorder is a disorder caused by SARS-Cov2, typically COVID-19.
#4. The BTN3A activating antibody for use according to any one of #1-#3, wherein said BTN3A activating antibody binds to BTN3A with a K_{D} of 10 nM or less, preferably with a K_{D} of 1 nM or less, as measured by SPR, for example as described in the Examples.
#5. The BTN3A activating antibody for use according to any one of #1-#4, wherein said BTN3A activating antibody has one or more of the following properties:
   (i) it binds to human BTN3A1 with a K_{D} of 10 nM or less, preferably with a K_{D} of 5 nM or less, or with a K_{D} of 1 nM or less, as measured by surface plasmon resonance (SPR) for example as described in the Examples;
   (ii) it binds to human PBMCs with an EC₅₀ of 50 μg/ml or below, preferably of 10 μg/ml or below, as measured in a flow cytometry assay, for example as described in the Examples; and/or,
   (iii) it induces the activation of γδ T cells, typically Vγ9Vδ2 T cells, in co-culture with BTN3A expressing cells, with an EC50 below 5 μg/ml, preferably of 1 μg/ml or below, as measured in a degranulation assay, for example as described in the Examples.
#6. The BTN3A activating antibody for use according to any one of #1-#5, wherein said BTN3A antibody has one or more the following properties:
   (i) it potentiates in vitro the reduction of C. burnetii bacterial load of monocytes in the presence of Vγ9Vδ2 T cells,
   (ii) it increases in vitro cytotoxic activity of Vγ9Vδ2 T cells towards C. burnetii infected monocytes, typically after 4 hours of co-culture.
#7. The BTN3A activating antibody for use according to any one of #1-#5, wherein said BTN3A activating antibody increases in vitro cytotoxic activity of Vγ9Vδ2 T cells towards SARS-Cov2 infected cells, for example as measured in vitro in co-cultures of infected cells with Vγ9Vδ2 T cells.
#8. The BTN3A activating antibody for use according to any one of #1-#7, wherein said BTN3A activating antibody comprises HCDRs1-3 of SEQ ID NO:5-7 and LCDRs1-3 of SEQ ID NO:8-10.
#9. The BTN3A activating antibody for use according to any one of #1-#7, wherein said BTN3A activating antibody comprises HCDRs1-3 of SEQ ID NO:11-13 and LCDRs1-3 of SEQ ID NO: 14-16.
#10. The BTN3A activating antibody for use according to any one of #1-#7, wherein said BTN3A antibody activating comprises HCDRs1-3 of SEQ ID NO: 17-19 and LCDRs1-3 of SEQ ID NO: 20-22
#11. The BTN3A activating antibody for use according to any one of Claims 1-10, wherein said BTN3A activating antibody either:
   comprises (a) a variable heavy chain (VH) polypeptide comprising an amino acid sequence that is at least about 95%, 96%, 97%, 98%, 99% or 100% identical to the amino acid sequence of SEQ ID NO:1, and (b) a variable light chain (VL) polypeptide comprising an amino acid sequence that is at least about 95%, 96%, 97%, 98%, 99% or 100% identical to of SEQ ID NO: 2;
   comprises (a) a variable heavy chain (VH) polypeptide comprising an amino acid sequence that is at least about 95%, 96%, 97%, 98%, 99% or 100% identical to the amino acid sequence of SEQ ID NO:3, and (b) a variable light chain (VL) polypeptide comprising an amino acid sequence that is at least about 95%, 96%, 97%, 98%, 99% or 100% identical to of SEQ ID NO: 4;
   comprises (a) a variable heavy chain (VH) polypeptide comprising an amino acid sequence that is at least about 95%, 96%, 97%, 98%, 99% or 100% identical to the amino acid sequence of SEQ ID NO:63, and (b) a variable light chain (VL) polypeptide comprising an amino acid sequence that is at least about 95%, 96%, 97%, 98%, 99% or 100% identical to of SEQ ID NO: 64;
   competes for binding to BTN3A1 with mAb 20.1 as produced by the hybridoma deposited at the CNCM under deposit number I-4401; or,
   competes for binding to BTN3A1 with mAb 7.2 as produced by the hybridoma deposited at the CNCM under deposit number 1-4402.
#12. The BTN3A activating antibody for use according to any of #1-#11, wherein said BTN3A activating antibody comprises a variable heavy chain VH of SEQ ID NO:1 and a light chain VL of SEQ ID NO:2.
#13. The BTN3A activating antibody for use according to any one of #1-#12, wherein said BTN3A antibody comprises a mutant or chemically modified IgG1 constant region, wherein said mutant or chemically modified IgG1 constant region confers no or decreased binding to Fcγ receptors when compared to a corresponding antibody with wild type IgG1 isotype constant region.
#14. The BTN3A activating antibody for use according to #13, wherein said mutant IgG1 constant region is the IgG1 triple mutant L247F L248E and P350S.
#15. The BTN3A activating antibody for use according to any one of #1-#8, and #11-#14, wherein the BTN3A antibody comprises a heavy chain of SEQ ID NO: 23 and a light chain of SEQ ID NO: 24.
#16. The BTN3A activating antibody for use according to any one of #1-#15, wherein said BTN3A activating antibody is administered in combination simultaneously, sequentially or separately with a cytokine for in vivo Vγ9Vδ2 T cell proliferation, for example IL2 or IL15 cytokine or their pegylated derivatives.
#17. The BTN3A activating antibody for use according to any one of #1-#16, for treating SARS-Cov2 viral infectious disorder, wherein said anti-BTN3A antibody is administered in combination, simultaneously or separately with an antiviral or anti-inflammatory treatment, preferably selected from the group consisting of remdesivir, baricitinib, bamlanivimab, bamlanivimab/etesevimab, casirivimab/imdevimab, dexamethasone, budesonide and tocilizumab.
#18. The BTN3A activating antibody for use according to any one of #1-#17, for treating SARS-Cov2 viral infectious disorder, wherein said subject is a human subject which has been diagnosed as being SARS-Cov2 positive.
#19. The BTN3A activating antibody for use according to any one of #1-#18, for treating SARS-Cov2 viral infectious disorder, wherein said subject is a human subject which has mild or moderate COVID-19.
#20. The BTN3A activating antibody for use according to any one of #1-#18, for treating SARS-Cov2 viral infectious disorder, wherein said subject is at high risk of progressing to severe COVID-19.
#21. The BTN3A activating antibody for use according to any one of #1-#18, for treating SARS-Cov2 viral infectious disorder, wherein said subject is having severe COVID-19.
#22. The BTN3A activating antibody for use according to any one of #1-#15, for treating a disorder caused by Coxiella Burnetii infection, wherein said BTN3A antibody is administered in combination, simultaneously or separately with an antibacterial treatment selected from antibiotics, preferably selected from doxycycline, tetracycline, chloramphenicol, ciprofloxacin, ofloxacin, and hydroxychloroquine.
#23. The BTN3A activating antibody for use according to any one of #1-#15 and #22, for treating a disorder caused by Coxiella Burnetii infection, wherein said subject has been diagnosed as being positive for Coxiella Burnetii infection.
#24. The BTN3A activating antibody for use according to any one of #1-#15 and #22-#23, for treating a disorder caused by Coxiella Burnetii infection, wherein said subject has Q fever.
#25. The BTN3A activating antibody for use according to any one of #1-#24, wherein said BTN3A antibody is administered to the subject in need thereof, by intravenous infusion, for example at a dose comprised between 1 mg and 1 g, for example between 1 mg and 200 mg.

### Examples

### Assays for Testing Functional Properties of Anti-BTN3A Activating Antibodies

### 1. Assay for Determining Binding Affinity by SPR of a BTN3A Activating Antibody

Multi-cycle kinetic analysis can be performed any BTN3A antibody candidate using a Biacore T200 (serial no. 1909913) instrument running Biacore T200 Evaluation Software V2.0.1 (Uppsala, Sweden).

Purified antibodies are diluted to a concentration of 2 μg/ml in 2% BSA/PBS. At the start of each cycle, each antibody is captured on the Protein A at a density (RL) of ˜ 146.5 RU (theoretical value to obtain an RMax of ˜50 RU). Following capture, the surface is allowed to stabilize before injection of the BTN3A1 antigen (Sino Biological cat. no. 15973-H08H). BTN3A1 is titrated in 0.1% BSA/HBS-P+ (running buffer) in a two-fold dilution range from 25 to 0.78 nM. The association phase is monitored for 400 seconds and the dissociation phase for 35 minutes (2100 seconds). Kinetic data are obtained using a flow rate of 50 μl/min to minimize any potential mass transfer effects. Regeneration of the Protein A surface is conducted using two injections of 10 mM glycine-HCL pH 1.5 at the end of each cycle. Two blanks (no BTN3A1) and a repeat of a single concentration of the analyte were performed for each tested antibody to check the stability of the surface and analyte over the kinetic cycles. The signal from the reference channel Fc1 is subtracted from that of Fc2, Fc3 and Fc4 to correct for differences in non-specific binding to a reference surface. Additionally, blank runs are subtracted for each Fc to correct any antigen-independent signal variation, such as drift. Sensorgrams were fitted using a one-to-one binding mathematical model with a global RMax parameter and no bulk signal (Constant RI=0 RU).

### 2. Assay for Determining Binding by Flow Cytometry on Human PBMCs of a BTN3A Activating Antibody

The BTN3A activating antibodies for use according to the present disclosure can be characterized for their binding to BTN3A as expressed in human PBMCs, isolated from blood of healthy donors. PBMCs are isolated from buffy coats using Lymphoprep (Axis-shield, Dundee, UK) density centrifugation. PBMCs are then frozen and stored at −80° C. or in liquid nitrogen until required.

100 μl cells at 1×10⁶ cells/ml are transferred to each well of a fresh U-shaped bottom 96-well plate, then the plate is centrifuged and supernatant discarded.

A serial dilution of the antibodies, 0.001 μg/ml to 150 μg/ml is prepared in PBS 2 mM EDTA. Human PBMCs are resuspended in 50 μl of the diluted test antibody titration series prepared.

After incubation for 30 minutes at 4° C. in the dark, the plate is centrifuged and washed twice with 150 μl/well of PBS 2 mM EDTA following which the wells are resuspended in 50 μl of a mix composed of goat anti-human antibody (PE labelled) diluted 1/100 and Live/dead neat IR diluted 1/500 in PBS 2 mM EDTA.

After incubation for 15 minutes at 4° C. in the dark, the plate is centrifuged and washed once with 150 μl/well PBS 2 mM EDTA following which the wells are resuspended in 200 μl PBS 2 mM EDTA. Cells are analyzed on a BD LSR Fortessa Cytometer. Data are analyzed using a FlowJo software (Version 10, FlowJo, LLC, Ashland, USA) (Data not shown).

Same protocol can be performed to test binding on cynomolgus BTN3A as expressed from cynomolgus PBMCs and on Daudi Burkitt's lymphoma cell line.

### 3. In Vitro Functional Efficacy: γδ-T Cell Degranulation Assay

The assay consists of measuring activating effect of BTN3A antibodies on γδ-T cell degranulation against Daudi Burkitt's lymphoma cell line (Harly et al., 2012). γδ-T cells are expanded from PBMCs of healthy donors by culturing with zoledronic acid (1 μM) and IL2 (200 Ui/ml) for 11-13 days. IL2 is added at day 5, day 8 and every 2 days thereafter. The percentage of γδ-T cells is determined at the initiation of culture and assessed for the time of culture by flow cytometry until it reached at least 80%. Frozen or fresh γδ-T cells are then used in degranulation assays against Daudi cell line (E:T ratio of 1:1), whereby the cells are co-cultured for 4 hours at 37° C. in presence of 10 μg/ml of the 7.2 and 20.1 humanized variants and their chimeric versions. Activation by PMA (20 ng/ml) plus lonomycin (1 μg/ml) served as positive control for γδ-T cell degranulation, and medium alone as negative control. At the end of 4 hours co-incubation, cells are analyzed by flow cytometry to evaluate the percentage of γδ-T cells positive for CD107a (LAMP-1, lysosomal-associated membrane protein-1)+CD107b (LAMP-2). CD107 is mobilized to the cell surface following activation-induced granule exocytosis, thus measurement of surface CD107 is a sensitive marker for identifying recently degranulated cytolytic T cells.

4. Assay for Determining Cytotoxic Activity of Vγ9Vδ2 T Cells Towards Infected Cells with SARS-Cov2 in Presence of a BTN3A Activating Antibody

Monocytes, MDMs, BEAS-2B and MRC-5 are labeled with 10 μM Cell Proliferation Dye eFluor® 670 (Invitrogen) and then stimulated with virus. Target cells are co-cultured with Vγ9Vδ2 T cells (effector) at effector-to-target (E:T) ratio of 1:1 in presence of a candidate anti-BTN3A activating antibody or control mAb 20.1 (0, 0.1, 1 or 10 μg/ml). After 24 hours, cells are stained with CellEvent Caspase-3/7 Green (Invitrogen) to identify dead cells. The cytotoxicity is assessed by flow cytometry as the percentage of Caspase 3/7+ cells in the target cell population. Data are collected on a BD Canto II instrument (BD Biosciences) and analyzed with FlowJo software (FlowJo v10.6.2).

5. Assay for Determining IFN-γ Secretion of Vγ9Vδ2 T Cells Towards Infected Cells with SARS-Cov2 in Presence of a BTN3A Activating Antibody

Monocytes, MDMs, BEAS-2B and MRC-5 are stimulated with virus and co-cultured with Vγ9Vδ2 T cells (effector) at effector-to-target (E:T) ratio of 1:1 in presence of candidate anti-BTN3A activating antibody or control mAb 20.1 (0, 0.1, 1 or 10 μg/ml). After 24 hours, culture supernatants from co-cultures are collected, and IFN-γ secretion is detected with the human IFN-γ Immunoassay Kit (R&D Systems) according to the manufacturer's instructions.

6. Assay for Determining Degranulation of Vγ9Vδ2 T Cells Towards Infected Cells with Coxiella burnetii in Presence of a BTN3A Activating Antibody

Monocytes infected by C. burnetii are co-cultured with Vγ9Vδ2 T cells at effector-to-target (E:T) ratio of 1:1 in presence of a candidate anti-BTN3A activating antibody or control mAb20.1, and fluorochrome-labeled CD107a and CD107b (BD Biosciences). After 4 hours, cells are harvested and stained with fluorochrome-labeled TCR-specific mAbs (Miltenyi Biotec) and a viability marker (Live/Dead Near IR, Invitrogen). The degranulation is evaluated by flow cytometry as the percentage CD107a/b⁺ cells in the γδ T cell population. Data are collected on a Navios instrument (Beckman Coulter) and analyzed with FlowJo software (FlowJo v10.6.2).

7. Assay for Determining Cytotoxic Activity of Vγ9Vδ2 T Cells Towards Infected Cells with Coxiella burnetii in Presence of a BTN3A Activating Antibody

Monocytes infected by C. burnetii are labeled with 10 μM Cell Proliferation Dye eFluor® 670 (Invitrogen) and then co-cultured with Vγ9Vδ2 T cells at E:T ratio of 1:1 in presence of a candidate anti-BTN3A activating antibody or control mAb20.1. After 4 hours, cells were stained with CellEvent Caspase-3/7 Green (Invitrogen) to identify dead cells. The cytotoxicity was assessed by flow cytometry as the percentage of Caspase 3/7⁺ cells in the target cell population. Data were collected on a BD Canto II instrument (BD Biosciences) and analyzed with FlowJo software (FlowJo v10.6.2).

### Example 1: Humanization of mAb 20.1 and Characterization

### 1. Description of Humanization Strategies

a. Design of Composite Human Antibody™ Variable Region Sequences

Structural models of the murine 7.2 and 20.1 antibody V regions were produced using Swiss PDB and analyzed in order to identify important “constraining” amino acids in the V regions that were likely to be essential for the binding properties of the antibodies. Most residues contained within the CDRs (using both Kabat and Chothia definitions) together with a number of framework residues were considered to be important. From the above analysis, Composite Human sequences of 7.2 and 20.1 antibodies have been created.

b. CD4+ T Cell Epitope Avoidance

Based upon the structural analysis, a large preliminary set of sequence segments that could be used to create 7.2 and 20.1 humanized variants were selected and analyzed using iTope™ technology for in silico analysis of peptide binding to human MHC class II alleles (Perry et al., 2008), and using the TCED™ of known antibody sequence-related T cell epitopes (Bryson et al., 2010). Sequence segments that were identified as significant non-human germline binders to human MHC class II or that scored significant hits against the TCED™ were discarded. This resulted in a reduced set of segments, and combinations of these were again analyzed, as above, to ensure that the junctions between segments did not contain potential T cell epitopes. Selected sequence segments were assembled into complete V region sequences predicted to be devoid of significant T cell epitopes. Several heavy chains and light chains sequences were then chosen for gene synthesis and expression in mammalian cells for mAbs 7.2 and 20.1.

### 2. Generation of Humanized Variants and Preliminary Characterization

a. Construction of Humanized Variants Plasmids

7.2 and 20.1 humanized variants were synthesized with flanking restriction enzyme sites for cloning into an expression vector system for human IgG4 (S241P, L248E) heavy and kappa light chains. All constructs were confirmed by sequencing.

b. Expression of Antibodies

Chimeric 7.2 and 20.1 (VHO/VKO), two control combinations (VHO/Vκ1, VH1/Vκ0) and combinations of humanized heavy and light chains were transiently transfected into FreeStyle™ CHO-S cells (ThermoFisher, Loughborough, UK) using a MaxCyte STX® electroporation system (MaxCyte Inc., Gaithersburg, USA) from corresponding endotoxin-free DNA. Transfections were undertaken for each antibody using OC-400 processing assemblies. Following cell recovery, cells were diluted to 3×10⁶ cells/mL into CD Opti-CHO medium (ThermoFisher, Loughborough, UK) containing 8 mM L-Glutamine (ThermoFisher, Loughborough, UK) and 1× Hypoxanthine-Thymidine (ThermoFisher, Loughborough, UK). 24 hours post-transfection, the culture temperature was reduced to 32° C. and 1 mM sodium butyrate (Sigma, Dorset, UK) was added. Cultures were fed daily by the addition of 3.6% (of the starting volume) feed (2.5% CHO CD Efficient Feed A (ThermoFisher, Loughborough, UK), 0.5% Yeastolate (BD Biosciences, Oxford, UK), 0.25 mM Glutamax (ThermoFisher, Loughborough, UK) and 2 g/L Glucose (Sigma, Dorset, UK)). IgG supernatant titers were monitored by IgG ELISA and transfections were cultured for up to 14 days prior to harvesting supernatants.

c. Selection of mAb 3, a Humanized Form of mAb 20.1

2 humanized variants of mAb 20.1 were selected out of 20 humanized candidates for further characterization.

The table 4 below summarizes the comparative data between the murine parent antibodies mAb 7.2, mAb 20.1, and a humanized version of mAb 20.1 having the VH of SEQ ID NO: 1 and VL of SEQ ID NO:2.

TABLE 4
Functional properties of humanized mAb 20.1

| | 7.2 Parent | 20.1 Humanized | 20.1 Parent |
|---|---|---|---|
| Candidate | murine Ab | mAb 3 | murine Ab |
| Biacore affinity | 1.76 | 3.19 | 2.34 |
| Multi-Cycle | | | |
| Kinetics(×10⁻¹⁰) | | | |
| (K_{D}, M) | | | |
| Binding on human PBMC | 3.6 | 3.38 | 3.02 |
| (EC₅₀, μg/mL) | | | |
| Binding | 7.75 | 5.74 | 4.06 |
| Cyno PBMC | | | |
| (EC₅₀, μg/mL) | | | |
| Binding to lymphoma | 1.44 | 1.59 | 1.19 |
| (Daudi)(EC₅₀, μg/mL) | | | |
| Functional assay (Daudi) | 0.02 | 0.02 | 0.02 |
| (γδT cell-based, | | | |
| EC₅₀, μg/mL) | | | |
| Functional assay | 0.12 | 0.15 | 0.05 |
| (AML sensitive) | | | |
| (γδT cell-based, | | | |
| EC₅₀, μg/mL) | | | |
| Functional assay | 0.42 | 0.32 | 0.18 |
| (AML resistant) | | | |
| (γδT cell-based, | | | |
| EC₅₀, μg/mL) | | | |

### Example 2: Evidence for Use of Activating BTN3A Antibody for Treating SARS-Cov2 Infection

### Material and Methods

### Cell Isolation and Cell Lines Cultures

Blood samples from 15 healthy volunteers were obtained from local blood bank (agreement N°7828, “Etablissement Français du Sang”, Marseille, France). Peripheral blood mononuclear cells (PBMCs) were isolated by density gradient centrifugation from buffy coats (by density gradient centrifugation using Ficoll (Eurobio, Les Ulis, France).

Monocytes were purified from PBMCs through a CD14 selection using MACS magnetic beads (Miltenyi Biotec, Bergisch Glabach, Germany) and cultured in Roswell Park Memorial Institute-1640 medium (RPMI, Life Technologies, Carlsbad, CA, USA) containing 10% fetal bovine serum (FBS, Gibco, Life Technologies), 2 mM L-glutamine, 100 U/mL penicillin and 50 μg/mL streptomycin (Life Technologies). For macrophages derived from monocytes (MDMs), cells were cultured in RPMI-1640 containing 10% inactivated human AB-serum (MP Biomedicals, Solon, OH), 2 mM glutamine, 100 U/mL penicillin and 50 μg/mL streptomycin during. After 3 days, the medium was replaced by RPMI-1640 containing 10% FBS and 2 mM glutamine, and cells were differentiated into macrophages for 4 additional days.

Vγ9Vδ2 T cells were expanded from fresh PBMCs. Briefly, PBMCs were grown in RPMI-1640 medium supplemented with 10% FBS, interleukin-2 (IL-2, to a final concentration of 200 UI/ml) and Zoledronic acid monohydrate (final concentration of 1 μM). IL-2 was added every 2 days beginning on day 5. After being cultured for 12 days, the purity of the Vγ9Vδ2 T cells was assessed by flow cytometry analysis and then frozen. Vγ9Vδ2 T cells were further purified and concentrated to a purity of up to 98% using the TCRγ/δ+ T Cell Isolation Kit (Miltenyi Biotec).

Normal human bronchial epithelial cells (BEAS-2B cells, ATCC® CRL-9609™) were cultured in LHC-9 medium (Life Technologies) and normal human lung fibroblast (MRC-5 cells, ATCC® CCL-171™) were cultured in Minimum Essential Media (MEM, Life Technologies) supplemented with 4% FBS and 2 mM L-glutamine at 37° C. in an atmosphere of 5% CO₂.

### Virus Production and Cell Infection

SARS-CoV-2 strain IHU-MI6 was obtained after Vero E6 cells (ATCC® CRL-1586™) infection in MEM supplemented with 4% FBS as previously described (Boumaza et al., 2020). Monocytes and MDMs isolated from PBMCs of healthy donors and BEAS-2B and MRC-5 cells were infected with virus suspension at multiplicity of infection (MOI) of 1 for 24 hours at 37° C. in the presence of 5% CO₂ and 95% air in a humidified incubator.

RNA Isolation and qRT-PCR

Total RNA was extracted from cells (2.10⁶ cells/well) using the RNeasy Mini Kit (Qiagen, Courtaboeuf, France) with DNase I treatment to eliminate DNA contaminants as previously described (Mezouar et al., 2019c). The quality and quantity of the extracted RNAs were evaluated using a NanoDrop spectrophotometer (Nanodrop Technologies, Wilmington, USA). Reverse transcription of isolated RNA was performed using a Moloney murine leukemia virus-reverse transcriptase kit (Life Technologies) and oligo (dT) primers. To quantify BTN3A expression levels, real time q-PCR was performed using Smart SYBR Green fast Master kit (Roche Diagnostics, Meylan, France) and specific primers (Table 5). To quantify BTN2A expression levels, real time q-PCR was performed using TaqMan® Fast Advanced Master Mix (Applied Biosystems, Life Technologies) and specific probes (Table 5). All q-PCRs were performed using a CFX Touch Real-Time PCR Detection System (Bio-Rad). The results were normalized using the ACTB or GAPDH housekeeping genes and are expressed as relative expression of investigated genes where ΔCt=Ct Target−Ct housekeeping gene as previously described (Mezouar et al., 2019a). The threshold cycle (Ct) was defined as the number of cycles required to detect the fluorescent signal.

TABLE 5
Primers

| Gene | Forward primer (5′-3′) | Reverse primer (5′-3′) |
|---|---|---|
| ACTB | GGAAATCGTGCGTGACATTA | AGGAGGAAGGCTGGAAGAG |
| | (SEQ ID NO: 35) | (SEQ ID NO: 36) |
| BTN3A1 | TTCCAGGTCATAGTGTCTGC | TGAGCAGCTGAGCAAAAGG |
| | (SEQ ID NO: 37) | (SEQ ID NO: 38) |
| BTN3A2 | TGGGAATACCAAGGGA | AGTGAGCAGCTGGACCAAGA |
| | (SEQ ID NO: 39) | (SEQ ID NO: 40) |
| BTN3A3 | GAGGGAATACTAAGAAATGGT | GAAGAGGGAGACATGAAAGT |
| | (SEQ ID NO: 41) | (SEQ ID NO: 42) |

| | |
|---|---|
| GAPDH | Hs02786624_g1 (Thermo Fisher Scientific) |
| BTN2A1 | Hs00924832_m1 (Thermo Fisher Scientific) |
| BTN2A2 | Hs00950165_g1 (Thermo Fisher Scientific) |

### Flow Cytometry Staining and Data Acquisition and Analysis

For analysis of cells infected by SARS-CoV-2 in vitro, cells were suspended in phosphate buffer saline (Life Technologies) containing 1% FBS and 2 mM EDTA (Sigma-Aldrich). Cells were labeled with viability dye (LIVE/Dead Near IR, Invitrogen) with anti-BTN3A (103.2) or anti-BTN2A (7.48) mAb or with the isotype control (Miltenyi Biotech). After 30 min incubation, primary antibodies binding was detected with Alexa Fluor 488 anti-mouse (Invitrogen). Data were collected on a BD Canto II instrument (BD Biosciences) and analyzed with FlowJo software (FlowJo v10.6.2, Ashland, OR).

Viral RNA Extraction and qRT-PCR

Viral RNA was extracted from infected cells using NucleoSpin® Viral RNA Isolation kit (Macherey-Nagel, Hoerdt, France) following the manufacturer's recommendations. Virus detection was performed using One-Step RT-PCR SuperScript™ III Platinum™ Kit (Life Technologies). Thermal cycling was achieved at 55° C. for 10 minutes for reverse transcription, pursued by 95° C. for 3 minutes and then 45 cycles at 95° C. for 15 seconds and 58° C. for 30 seconds using a LightCycler 480 Real-Time PCR system (Roche, Rotkreuz, Switzerland). The primers and the probes were designed against the E gene (Boumaza et al., 2020).

### Cytotoxicity Assay

Monocytes, MDMs, BEAS-2B and MRC-5 were labeled with 10 μM Cell Proliferation Dye eFluor® 670 (Invitrogen) and then stimulated with virus. Target cells were co-cultured with Vγ9Vδ2 T cells (effector) at effector-to-target (E:T) ratio of 1:1 in presence of anti-BTN3A 20.1 mAb (0, 0.1, 1 or 10 μg/ml). After 24 hours, cells were stained with CellEvent Caspase-3/7 Green (Invitrogen) to identify dead cells. The cytotoxicity was assessed by flow cytometry as the percentage of Caspase 3/7+ cells in the target cell population. Data were collected on a BD Canto II instrument (BD Biosciences) and analyzed with FlowJo software (FlowJo v10.6.2).

### IFN-γ Secretion Assay

Monocytes, MDMs, BEAS-2B and MRC-5 were stimulated with virus and co-cultured with Vγ9Vδ2 T cells (effector) at effector-to-target (E:T) ratio of 1:1 in presence of anti-BTN3A 20.1 mAb (0, 0.1, 1 or 10 μg/ml). After 24 hours, culture supernatants from co-cultures were collected, and IFN-γ secretion was detected with the human IFN-γ Immunoassay Kit (R&D Systems) according to the manufacturer's instructions.

### Statistical Analysis

Statistical analysis was performed with GraphPad Prism (version 8.0, La Jolla, CA), for transcriptional analysis using the Student t or nonparametric Mann-Whitney U test and for Spectral Cytometry using Kruskal-Wallis test, followed by Dunn's multiple comparisons and the Mann-Whitney test (limit of significance: p<0.05).

### Results

### Effects of SARS-CoV-2 Infection on BTN3A and BTN2A Expression

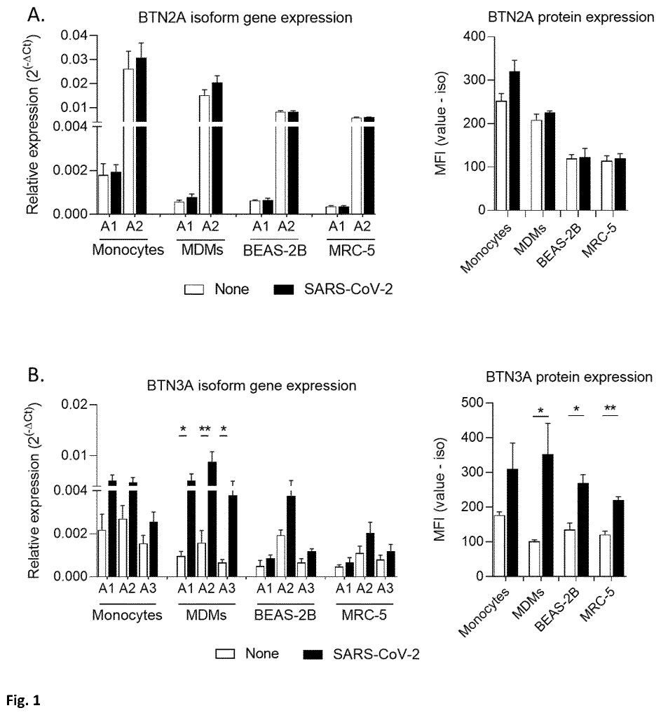

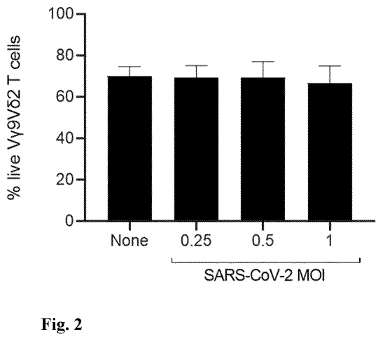

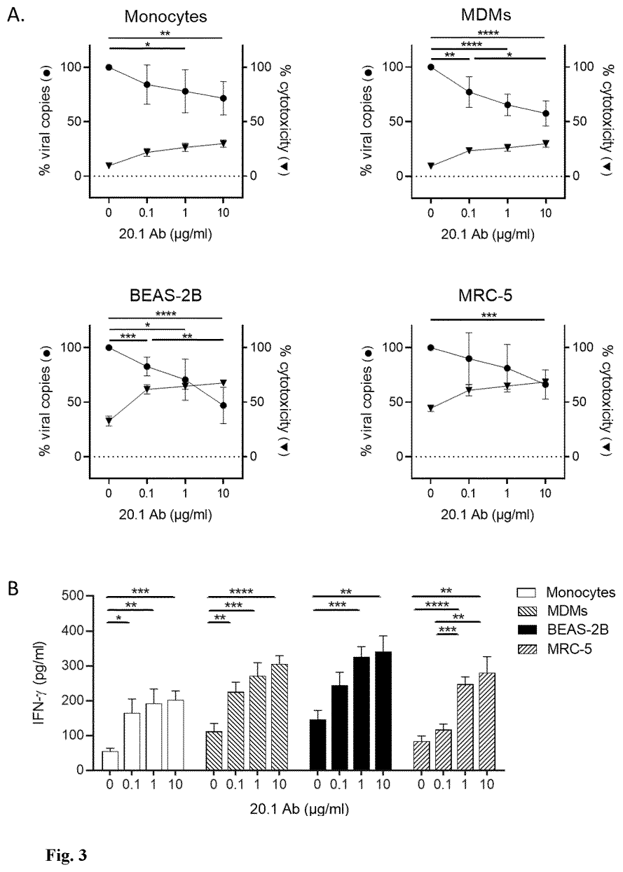

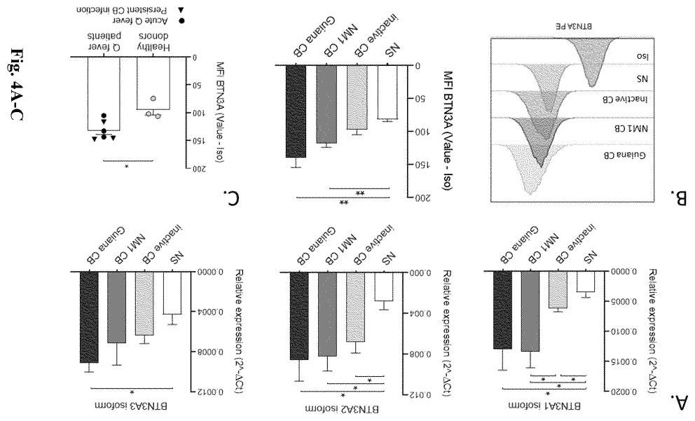

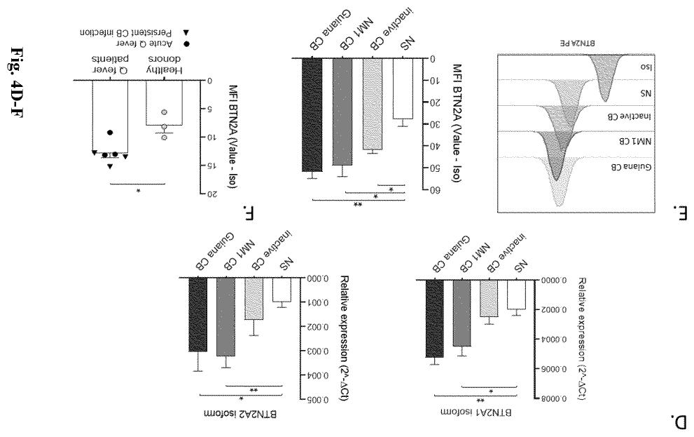

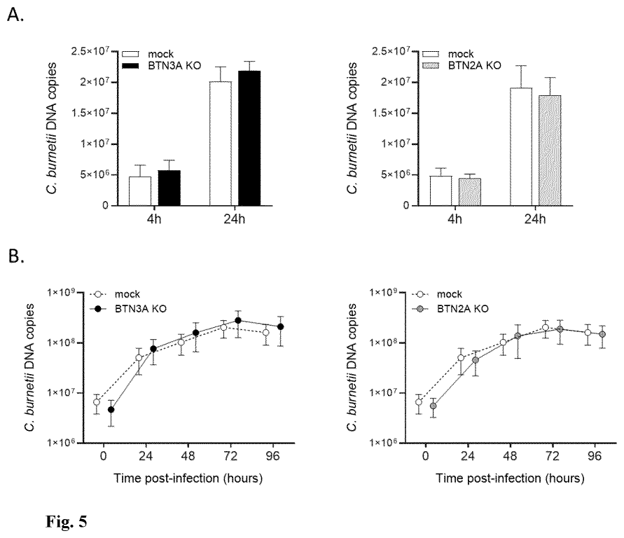

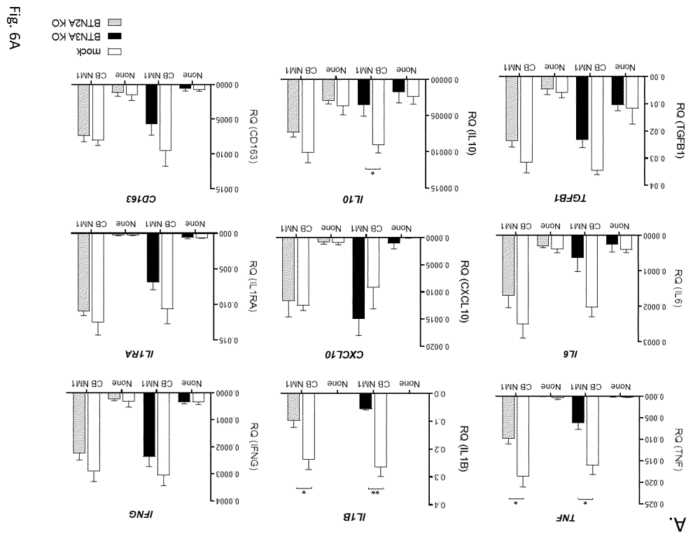

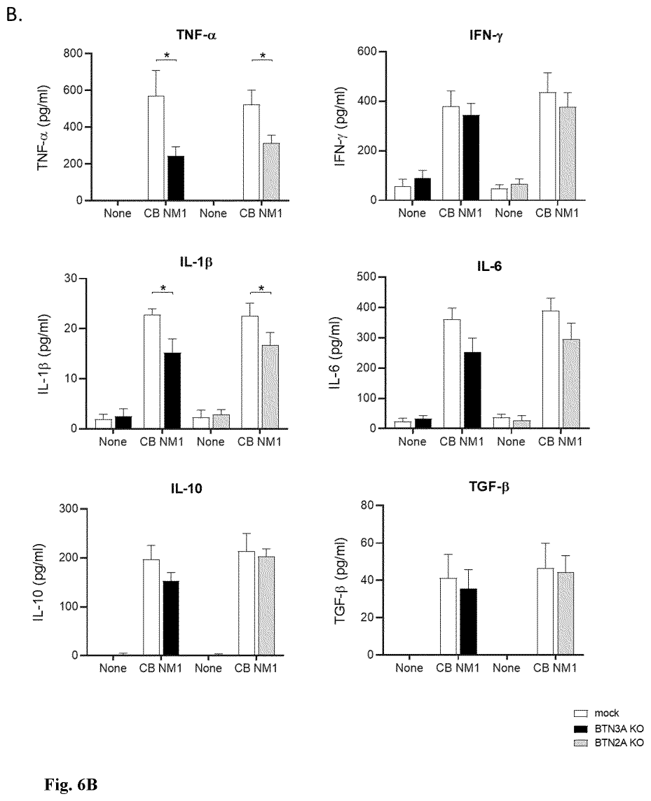

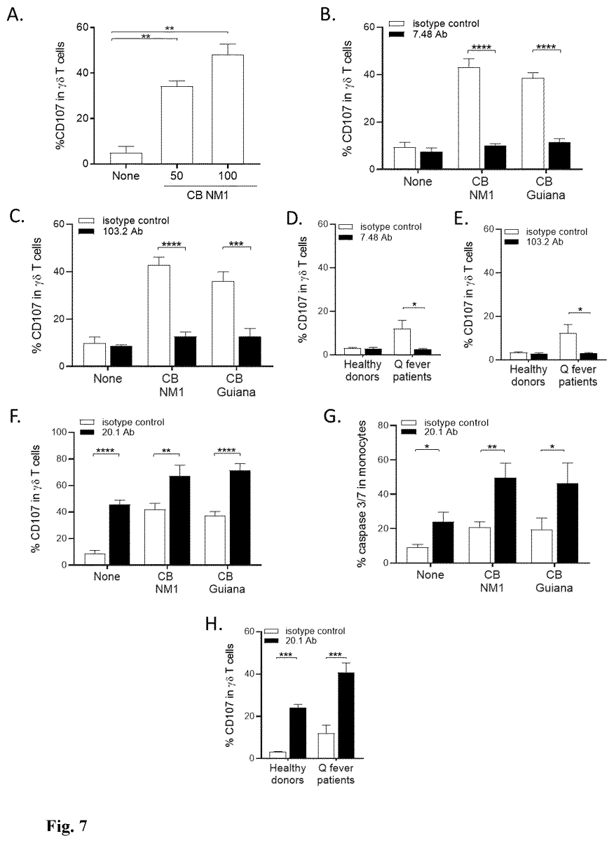

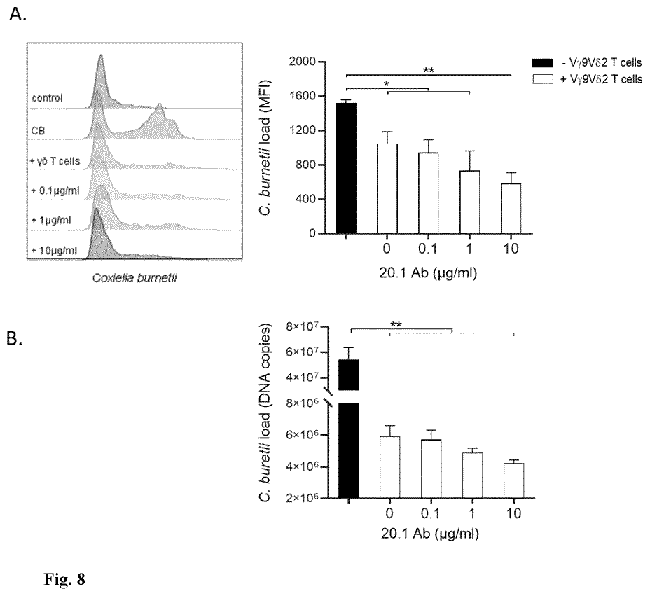

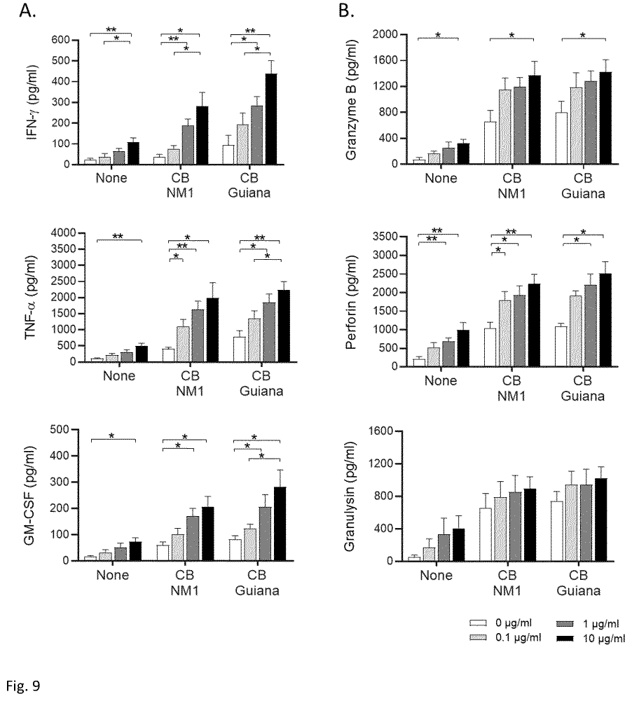

First, we evaluated whether SARS-CoV-2 infection affected the expression of BTN3A and BTN2A on cells from myeloid (primary cell cultures of monocytes or macrophage derived from monocytes (MDMs)) or lung origin (normal epithelial lung cell lines: BEAS-2B or MRC-5). No effects were observed on the transcriptional (isoform genes 2A1, 2A2) or protein expression of BTN2A (FIG. 1A). In contrast, gene expression of the three isoforms (3A1, 3A2, 3A3) of BTN3A was significantly increased in presence of SARS-CoV-2 in MDMs cultures in comparison to cultures without viruses. Indeed, the relative expression for the 3 isoforms of BTN3A was increased about 5-fold in SARS-CoV-2 stimulated MDMs compared to unstimulated MDMs (3A1 p=0.012, 3A2 p=0.006, 3A3 p=0.028) (FIG. 1B). No statistically significant differences of BTN3A transcripts were observed in monocytes or lung cells cultures. However, we observed a significant increase of BTN3A protein expression on MDMs, BEAS-2B, MRC-5 upon SARS-CoV-2 stimulation (FIG. 1B). BTN3A protein expression was increased 3.5-fold in MDMs and about 2-fold in BEAS-2B and MRC-5 cells following stimulation with SARS-CoV-2 (p=0.048, p=0.012 and p=0.002, respectively).

### Characterization of Vγ9Vδ2 T Cells Responses Against SARS-CoV-2 Infected Cells In Vitro

We then studied Vγ9Vδ2 T cells ability to inhibit SARS-CoV-2 replication in vitro, when activated with the reference anti-BTN3A monoclonal antibody 20.1. After confirming that the viral infection did not affect Vγ9Vδ2 T cells viability (FIG. 2), we co-cultured Vγ9Vδ2 T cells with myeloid or lung cells in presence of SARS-CoV-2 IHU-MI6 strain and increasing concentrations of the reference 20.1 mAb. The reference anti-BTN3A 20.1 produced a dose-dependent inhibition of viral replication (28.4% in monocytes cultures, 42.4% in MDMs, 33.7% in MRC-5 and 53.0% in BEAS-2B cultures at 10 μg/ml) (FIG. 3A). Cytotoxicity (caspase 3/7) of Vγ9Vδ2 T lymphocytes appeared to be higher against SARS-CoV-2 infected lung cells than against myeloid cells, and increased along with concentrations of the reference anti-BTN3A 20.1 antibody. The difference was statistically significant between doses of 0.1 and 10 g/ml in each of the four target cell cultures (Table 6).

Finally, we investigated if Vγ9Vδ2 T cells activated with the reference anti-BTN3A 20.1 exerted an IFN-γ mediated non-cytolytic anti-SARS-CoV-2 activity. Increasing concentrations of anti-BTN3A were associated with a significant and dose-dependent increase of IFN-γ in the supernatants of the four cell cultures (FIG. 3B and Table 6).

TABLE 6
Effect of the reference 20.1 mAb on Vγ9Vδ2 T
cell responses against SARS-CoV-2 infected cells

| | IFNg (pg/ml) | | | |
|---|---|---|---|---|
| | | | Mean ± SEM | |
| | isotype | 20.1 | difference | p |
| Monocytes | 54.89 | 202 | 147.2 ± 27.49 | 0.0003 |
| MDM | 111.7 | 306 | 194.3 ± 32.54 | 0.0001 |
| BEAS-2B | 146.5 | 340.7 | 194.2 ± 51 | 0.0038 |
| MRC-5 | 83.73 | 280.6 | 196.9 ± 47.88 | 0.0021 |

### Example 3: Evidence for use of activating anti-BTN3A antibody for treating Coxiella burnetii infection

### Material and Methods

### Cell Isolation

Blood samples (leucopacks) that we used in our study come from the French Blood Establishment (Etablissement Français du sang, EFS) that carries out donor inclusions, informed consent, and sample collection. Through a convention established between our laboratory and the EFS (No 7828), buffy coats were obtained, and peripheral blood mononuclear cells (PBMCs) were isolated as previously described.

Monocytes were purified from PBMCs through a CD14 selection using MACS magnetic beads (Miltenyi Biotec, Bergisch Glabach, Germany) and cultured in Roswell Park Memorial Institute-1640 medium (RPMI, Life Technologies, Carlsbad, CA, USA) containing 10% fetal bovine serum (FBS, Gibco, Life technologies), 2 mM L-glutamine, 100 U/mL penicillin and 50 μg/mL streptomycin (Life Technologies).

Vγ9Vδ2 T cells were expanded from fresh PBMCs as previously described. Briefly, PBMCs were grown in RPMI-1640 medium supplemented with 10% FBS, interleukin-2 (IL-2, to a final concentration of 200 UI/ml) and Zoledronic acid monohydrate (Zometa, to a final concentration of 1 μM). IL-2 was added every 2 days beginning on day 5 for 12 days and the purity of the Vγ9Vδ2 T cells was assessed by flow cytometry analysis (>85%) and then frozen at −80° C. in 10% dimethyl sulfoxide (Sigma-Aldrich, Saint-Quentin-Fallavier, France) and 90% FBS.

### Bacterial Production

Coxiella burnetii bacteria phase I (Nine Mile strain, RSA493 and Guiana strain, MST17) were cultured in L929 cells for 10 days. Briefly, infected cells were sonicated and centrifuged at 10,000×g for 10 minutes, then washed and stored at −80° C. The concentration of bacteria was determined using Gimenez staining, and bacterial viability was assessed using the Live/Dead BacLight bacterial viability kit (Molecular Probes, Eugene, OR, USA) following manufacturer's instructions.

### Bacterial Detection

DNA was extracted from C. burnetii infected cells using a DNA Mini Kit (Qiagen, Courtaboeuf, France). Infection was quantified by real time quantitative PCR (qPCR) performed with specific primers F (5′-GCACTATTTTTAGCCG-GAACCTT-3′ [SEQ ID NO:43]) and R (5′-TTGAGGAGAAAAACTGGATTGAGA-3′ [SEQ ID NO:44]) targeting the C. burnetii COM-1 gene, as previously described.

The presence of C. burnetii within cells was also assessed by flow cytometry. Briefly, infected cells were fixed with 4% paraformaldehyde and permeabilized with 0.1% Triton X-100 (Sigma-Aldrich). After washing, cells were incubated with an anti-rabbit directed against C. burnetii for 30 min and then with an Alexa 647 anti-rabbit antibody (Invitrogen). Data were collected on a BD Canto II instrument (BD Biosciences, Le Pont-de-Claix, France) and analyzed with FlowJo software (FlowJo v10.6.2, Ashland, OR).

RNA Isolation and qRT-PCR

Total RNA was extracted from cells (2.10⁶ cells/well) using the RNeasy Mini Kit (Qiagen) with Dnase I treatment to eliminate DNA contaminants as previously described. The quality and quantity of the extracted RNAs were evaluated using a NanoDrop spectrophotometer (Nanodrop Technologies, Wilmington, USA). Reverse transcription of isolated RNA was performed using a moloney murine leukemia virus-reverse transcriptase kit (Life Technologies) and oligo (dT) primers. The expression levels of genes involved in the M1/M2 response, as well as BTN3A isoform genes, was evaluated using real time qPCR, Smart SYBR Green fast Master kit (Roche Diagnostics, Meylan, France) and specific primers (Table 7). BTN2A expression levels was evaluated using real time qPCR, TaqMan® Fast Advanced Master Mix (Applied Biosystems, Life Technologies) and specific probes (Table 7). All qPCRs were performed using a CFX Touch Real-Time PCR Detection System (Bio-Rad, Marnes-la-Coquette, France). The results were normalized using the ACTB or GAPDH housekeeping gene and are expressed as relative expression of investigated genes with 2^{-ΔCt} where ΔCt=Ct_{target}−Ct_{housekeeping} gene as previously described. The threshold cycle (Ct) was defined as the number of cycles required to detect the fluorescent signal.

TABLE 7
Primers

| Gene | Forward primer (5′-3′) | Reverse primer (5′-3′) |
|---|---|---|
| ACTB | GGAAATCGTGCGTGACATTA | AGGAGGAAGGCTGGAAGAG |
| | (SEQ ID NO: 36) | (SEQ ID NO: 37) |

| | |
|---|---|
| GAPDH | Hs02786624_g1 |

M1 genes

| | | |
|---|---|---|
| TNF | AGGAGAAGAGGCTGAGGAACAAG | GAGGGAGAGAAGCAACTACAGACC |
| | (SEQ ID NO: 45) | (SEQ ID NO: 46) |
| IL1B | CAGCACCTCTCAAGCAGAAAAC | GTTGGGCATTGGTGTAGACAAC |
| | (SEQ ID NO: 47) | (SEQ ID NO: 48) |
| IL6 | CCAGGAGAAGATTCCAAAGATG | GGAAGGTTCAGGTTGTTTTCTG |
| | (SEQ ID NO: 49) | (SEQ ID NO: 50) |
| IFNG | GTTTTGGGTTCTCTTGGCTGTTA | ACACTCTTTTGGATGCTCTGGTC |
| | (SEQ ID NO: 51) | (SEQ ID NO: 52) |
| CXCL10 | TCCCATCTTCCAAGGGTACTAA | GGTAGCCACTGAAAGAATTTGG |
| | (SEQ ID NO: 53) | (SEQ ID NO: 54) |

M2 genes

| | | |
|---|---|---|
| IL10 | GGGGGTTGAGGTATCAGAGGTAA | GCTCCAAGAGAAAGGCATCTACA |
| | (SEQ ID NO: 55) | (SEQ ID NO: 56) |
| TGFB | GACATCAAAAGATAACCACTC | TCTATGACAAGTTCAAGCAGA |
| | (SEQ ID NO: 57) | (SEQ ID NO: 58) |
| IL1RA | TCTATCACCAGACTTGACACA | CCTAATCACTCTCCTCCTCTTCC |
| | (SEQ ID NO: 59) | (SEQ ID NO: 60) |
| CD163 | CGGTCTCTGTGATTTGTAACCAG | TACTATGCTTTCCCCATCCATC |
| | (SEQ ID NO: 61) | (SEQ ID NO: 62) |

BTN isoform genes

| | | |
|---|---|---|
| BTN3A1 | TTCCAGGTCATAGTGTCTGC | TGAGCAGCTGAGCAAAAGG |
| | (SEQ ID NO: 37) | (SEQ ID NO: 38) |
| BTN3A2 | TGGGAATACCAAGGGA | AGTGAGCAGCTGGACCAAGA |
| | (SEQ ID NO: 39) | (SEQ ID NO: 40) |
| BTN3A3 | GAGGGAATACTAAGAAATGGT | GAAGAGGGAGACATGAAAGT |
| | (SEQ ID NO: 41) | (SEQ ID NO: 42) |

| | |
|---|---|
| BTN2A1 | Hs00924832_m1 |
| BTN2A2 | Hs00950165_g1 |

### BTN3A and BTN2A Protein Expression

Cells were collected in PBS (Life Technologies) containing 1% FBS and 2 mM EDTA (Sigma-Aldrich) and labeled with viability dye (Live/Dead Near IR, Invitrogen), anti-BTN3A (clone 103.2) or anti-BTN2A (clone 7.48) mAbs or with the appropriate isotype control (Miltenyi Biotech). After 30 min incubation, primary antibody binding was detected with PE anti-mouse antibody (Invitrogen) and data were collected on a Navios instrument (Beckman Coulter) and analyzed with FlowJo software (FlowJo v10.6.2).

### Degranulation Assay

Monocytes were co-cultured with Vγ9Vδ2 T cells at effector-to-target (E:T) ratio of 1:1 in presence of anti-BTN2A mAb (clone 7.48) or anti-BTN3A mAb (clones 20.1 or 103.2) and fluorochrome-labeled CD107a and CD107b (BD Biosciences). After 4 hours, cells were harvested and stained with fluorochrome-labeled TCR-specific mAbs (Miltenyi Biotec) and a viability marker (Live/Dead Near IR, Invitrogen). The degranulation was evaluated by flow cytometry as the percentage CD107a/b⁺ cells in the γδ T cell population. Data were collected on a Navios instrument (Beckman Coulter) and analyzed with FlowJo software (FlowJo v10.6.2).

### Cytotoxicity Assay

Monocytes were labeled with 10 μM Cell Proliferation Dye eFluor® 670 (Invitrogen) and then co-cultured with Vγ9Vδ2 T cells at E:T ratio of 1:1 in presence of anti-BTN3A mAb (clone 20.1). After 4 hours, cells were stained with CellEvent Caspase-3/7 Green (Invitrogen) to identify dead cells. The cytotoxicity was assessed by flow cytometry as the percentage of Caspase 3/7⁺ cells in the target cell population. Data were collected on a BD Canto II instrument (BD Biosciences) and analyzed with FlowJo software (FlowJo v10.6.2).

### Immunoassays

Tumor necrosis factor-alpha (TNF-α), interferon-gamma (IFN-γ), Granulocyte-macrophage colony-stimulating factor (GM-CSF) (R&D Systems), granzyme B, perforin, and granulysin (Abcam) levels were quantified in the supernatants of monocyte/Vγ9Vδ2 T cells co-culture using specific immunoassay kits. TNF-α, IFN-γ, interleukin (IL)-1β, IL-6, IL-10 and transforming growth factor beta (TGF-β) (R&D Systems) levels were quantified in the supernatants of BTNKO cells following C. burnetii infection. The sensitivity of assays was 6.2 pg/mL for TNF-α, 5.7 pg/mL for IFN-γ, 1.0 pg/mL for IL-1β, 0.7 pg/mL for IL-6, 3.9 pg/mL for IL-10, 15.4 pg/mL for TGF-β, 3.0 pg/mL for GM-CSF, 20 μg/mL for granzyme B, 40 μg/mL for perforin and 10 μg/mL for granulysin.

### Statistical Analysis

Statistical analysis was performed with GraphPad Prism (8.0, La Jolla, CA), using the Mann-Whitney U test for transcriptional analysis and t test for flow cytometry and ELISA results. Hierarchical clustering of gene expression was analyzed using the ClustVis webtool. The limit of significance was set up at p<0.05.

### Results

C. burnetii Infection Modulates the Expression of BTN3A and BTN2A

To assess whether C. burnetii infection affected the expression of BTNs, monocytes from healthy donors were isolated and infected with the reference strain NM1 or with the Guiana strain described as more virulent. After 24 hours of C. burnetii NM1 strain infection, transcriptional expression increases of both BTN3A1 and BTN3A2 isoforms, but not of BTN3A3, were found. Guiana strain infection induced the three isoforms like (FIG. 4A). Interestingly, significant differences were observed between C. burnetii NM1 infection and the heat-inactivated form of this strain (p=0.0374), suggesting that virulence factor was involved in BTN3A1 expression. Significant increase of BTN3A protein expression was found in monocytes infected with C. burnetii NM1 and Guiana strains (p=0.0021 and p=0.0096, respectively) (FIG. 4B). Finally, BTN3A expression was assessed in PBMCs from Q fever patients with C. burnetii infection, in its active or persistent form. BTN3A protein expression was significantly higher in patients with Q fever compared to healthy donors (p=0.0185) with similar expression according to the disease form (FIG. 4C).

As BTN2A is involved in Vγ9Vδ2 T-cell activation, we also investigated whether C. burnetii infection affected its expression. After 24 hours of infection, BTN2A transcriptional expression for both isoforms (2A1 and 2A2) was significantly increased after C. burnetii NM1 and Guiana infection (2A1 p=0.0170 and p=0.0021, respectively; and 2A2 p=0.0054 and p=0.0463, respectively) compared to uninfected cells and without significant modulation compared to the heat-inactivated form (FIG. 4D). Regarding BTN2A protein expression, a significant increase was observed for C. burnetii infected monocytes (NM1 strain, p=0.0160; and Guiana strain, p=0.0018) compared to uninfected cells (FIG. 4E). Similarly to BTN3A, PBMCs from Q fever patients showed significantly higher expression of BTN2A compared to healthy donors (p=0.0125) without modulation according to the clinical form of the disease (FIG. 4F). Altogether, C. burnetii infection leads to increased expression of BTN3A and BTN2A of the host after in vitro infection as well as in samples from infected patients.

Involvement of BTN3A and BTN2A in C. burnetii Infection

Next, we investigated whether BTNs could be involved in the uptake or replication of C. burnetii. For this purpose, we performed a CRISPR-Cas9 knockout of the three BTN3A genes or the two BTN2A genes in the THP-1 cell line. Cells were transduced with guides targeting either BTN2A1 and BTN2A2 (BTN2AKO) or BTN3A1, BTN3A2 and BTN3A3 (BTN3AKO) isoforms or with an irrelevant CRISPR guide (mock). BTN3A- or BTN2A-invalidated (KO) cells were infected with C. burnetii NM1, and the bacterial load was assessed by qRT-PCR. No differences were observed on bacterial uptake (FIG. 5A) or replication (FIG. 5B) between BTN3AKO or BTN2AKO and mock cells (FIG. 5A) suggesting that BTN3A and BTN2A are not directly involved in the process of C. burnetii cell infection.

Involvement of BTN3A and BTN2A in the Inflammatory Response to C. burnetii Infection

We then investigated the involvement of BTNs in the host immune response following C. burnetii infection. As observed in the FIG. 3, C. burnetii infection results in modulation of both pro-inflammatory and anti-inflammatory genes in THP-1 cells. Upon infection with C. burnetii, the hierarchical clustering revealed a clustering dependent on cell type; BTN3AKO and BTN2KO cells were clustered apart from mock cells (FIG. 6A). BTN3AKO and BTN2KO cells presented a repression of inflammatory response following C. burnetii infection. Indeed, the expression of the inflammatory genes TNF and IL1B was significantly decreased by 3-fold and 2-fold in BTN3AKO and BTN2AKO cells, respectively, compared to mock cells (FIG. 6B). Furthermore, a decreased immunoregulatory response was observed after infection only in BTN3AKO cells. Indeed, the transcriptional expression of IL10 gene was significantly decreased by 2-fold compared to mock cells (p=0.0435) (FIG. 6B). Regarding the protein levels, BTN3AKO and BTN2AKO cells presented a significant decrease in TNF-α (50% and 30%, respectively) and IL-1B (up to 20%) release following C. burnetii infection compared to mock cells (FIG. 6C). No significant difference was observed in the levels of anti-inflammatory cytokines such as IL-10.

Taken together, these data reported that both BTN3A and BTN2A are involved in the inflammatory response to C. burnetii infection. The fact that the inflammatory response is repressed in the presence of only BTN3A or only BTN2A suggests that they may act independently.

C. burnetii Infection Leads to Vγ9Vδ2 Cell Activation in a BTN3A and BTN2A-Dependent Manner

As we showed that BTNs are over-expressed in monocytes following C. burnetii infection, we hypothesized that this process could enhance Vγ9Vδ2 T cell activation. After 4 hours of co-culture, C. burnetii infection of monocytes leads to increased degranulation (up to 5-fold), as depicted by an increased plasma membrane expression of CD107, with a dose-response according to the ratio bacteria:target cell (FIG. 7A). We then investigated whether this Vγ9Vδ2 T cell activation against C. burnetii-infected cells was dependent on BTNs using specific antagonist antibodies including anti-BTN3A antagonist (clone 103.2) and anti-BTN2A antagonist (clone 7.48). Both antibodies lead abrogation of Vγ9Vδ2 T cell degranulation after C. burnetii infection, indicating that both BTNs are involved in Vγ9Vδ2 T cell activation upon C. burnetii infection (FIGS. 7B and 7C). Interestingly, a trend to higher degranulation of Vγ9Vδ2 T cells was found in co-cultures of PBMCs from patients with Q fever compared to healthy donors (10% vs. 3%, p=0.173) without antibody treatment. This degranulation was also inhibited in the presence of anti-BTN3A 103.2 and anti-BTN2A 7.48 antagonist antibodies (FIGS. 7D and 7E). Taken together, C. burnetii infection leads to Vγ9Vδ2 T cell activation in a BTN3A and BTN2A dependent manner.

We next hypothesized that Vγ9Vδ2 T cell activation against C. burnetii-infected cells could be enhanced by a BTN3A activating antibody (clone 20.1), that mimics pAg-induced Vγ9Vδ2 T cell activation. As illustrated in the FIG. 7F and Table 8, we observed that the BTN3A activating antibody leads to increased expression of CD107 (FIG. 7F) and the cytotoxic activity (FIG. 7G) of Vγ9Vδ2 T cells towards C. burnetii-infected monocytes after 4 hours of co-culture. A similar effect was observed for all C. burnetii strains, suggesting that the reference 20.1 antibody can induce Vγ9Vδ2 T cell activation even towards more virulent bacteria. Finally, the use of the reference 20.1 antibody was also able to increase Vγ9Vδ2 T cell degranulation from PBMCs from C. burnetii patients and healthy donors (mean 24.13% and 3.243%, respectively, p=0.0002, n=3 each) (FIG. 7H). These data show that targeting Vγ9Vδ2 T cells with a BTN3A activating antibody leads to the activation of their effector functions.

TABLE 8
Effect of the reference 20.1 mAb on Vγ9Vδ2 T cell degranulation

| | Mean % CD107 + cells | | Mean ± SEM | |
|---|---|---|---|---|
| Strain | isotype | 20.1 | Difference | p |
| Non stimulated | 8.59 | 45.83 | 37.24 ± 2.06 | <0.0001 |
| CB NM1 | 42.1 | 67.2 | 25.1 ± 4.67 | 0.0017 |
| CB Guiana | 37.23 | 71.48 | 34.25 ± 2.96 | <0.0001 |

### BTN3A 20.1 Activating Antibody Increases Antimicrobial Activity of Vγ9Vδ2 T Cells

Since the reference BTN3A activating 20.1 antibody increases Vγ9Vδ2 T cell activation, we wondered whether it was able to boost their antimicrobial activity. For this purpose, monocytes were infected with C. burnetii NM1 for 24 hours and then co-cultured with Vγ9Vδ2 T cells for 4 hours in presence of 20.1 antibody (0, 0.1, 1 or 10 μg/ml) and the bacterial load was measured by flow cytometry and qPCR. Vγ9Vδ2 T cells co-incubation led to a significant reduction of C. burnetii load as depicted by a significant decreased MFI related to C.burnetti staining in co-cultures of infected monocytes and Vγ9Vδ2 T cells (FIG. 8A) and a decrease from 5.10⁷ to 6.10⁶ copies (p=0.0021) in co-cultures of infected monocytes and Vγ9Vδ2 T lymphocytes (FIG. 8B). The reference BTN3A activating 20.1 antibody resulted in a dose-dependent decrease in C. burnetii load in monocytes, reaching from 6.10⁶ to 4.2·10⁶ copies (0 vs. 10 μg/ml, respectively, p=0.0501) (FIG. 8B). Altogether, the reference BTN3A 20.1 activating antibody increases the antimicrobial activity of Vγ9Vδ2 T lymphocytes against monocytes infected with Coxiella burnetii.

### The Reference BTN3A 20.1 Antibody Increases the Secretion of Cytokines and Cytotoxic Molecules by Vγ9Vδ2 T Cells

Since the BTN3A activating antibody allows inhibition of bacterial load, we investigated whether this could be related to the secretion of cytokines and cytotoxic molecules, which are strongly produced by activated Vγ9Vδ2 T cells. Indeed, treatment of Vγ9Vδ2 T cell/C. burnetii-infected monocyte co-cultures by the reference 20.1 antibody increased in a dose-dependent manner the secretions of TFN-α, IFN-γ and GM-CSF (FIG. 9A, left panel). Moreover, a significant difference was observed between the 0.1 and 10 μg/ml doses for IFN-γ, TFN-α and GM-CSF secretion (p=0.0260, p=0.0443 and p=0.0265, respectively), in the case of infection with C. burnetii Guiana. Regarding cytotoxic molecules, granzyme B and perforin secretion was significantly higher in presence of 10 μg/ml 20.1 antibody in the case of monocytes infected with C. burnetii NM1 and Guina, and also for uninfected monocytes (FIG. 9B, right panel). Overall, the presence of the reference 20.1 antibody allows the increased secretion of cytokines and cytotoxic molecules (Table 9), both produced by the activated Vγ9Vδ2 T cells.

TABLE 9
Effect of the reference 20.1 mAb (10 μg/ml)
on Vγ9Vδ2 T cell cytotoxic response

| | | Mean (pg/ml); | | | |
|---|---|---|---|---|---|
| | | n = 4 | | Mean ± SEM | |
| | Strain | untreated | 20.1 | Difference | p |
| TNFa | CB NM1 | 414.3 | 1993 | 1578 ± 472.6 | 0.016 |
| | CB | 784.7 | 2239 | 1454 ± 323.5 | 0.004 |
| | Guiana | | | | |
| IFNg | CB NM1 | 37.73 | 281.8 | 244.1 ± 67.73 | 0.011 |
| | CB | 95.6 | 438.6 | 343 ± 78.05 | 0.0046 |
| | Guiana | | | | |
| GM-CSF | CB NM1 | 60.65 | 206.3 | 145.6 ± 41.56 | 0.0128 |
| | CB | 82.39 | 283.1 | 200.7 ± 65.76 | 0.022 |
| | Guiana | | | | |
| Granulysin | CB NM1 | 574 | 772 | 198 ± 0 | ns |
| | CB | 740 | 1024 | 284 ± 185.5 | ns |
| | Guiana | | | | |
| Granzyme | CB NM1 | 659.9 | 1375 | 715 ± 274.2 | 0.041 |
| B | CB | 798.3 | 1429 | 630.4 ± 254 | 0.047 |
| | Guiana | | | | |
| Perforin | CB NM1 | 1049 | 2242 | 1193 ± 294.4 | 0.007 |
| | CB | 709.5 | 1436 | 726.7 ± 257.6 | 0.030 |
| | Guiana | | | | |

### SEQUENCE LISTING

TABLE 10
(mAbs sequences and in particular CDR sequences are according to Kabat):

| SEQ | | | |
|---|---|---|---|
| ID | | Description of | |
| NO: | Type | the sequence | Sequence |
| 1 | aa | VH | QVQLVQSGAEVKKPGASVKVSCKASGYTFTRYYLYWVKQRPGQGL |
| | | Humanized | EWIGEINPNNGGTKFNEKFKSRATMTVDKSTRTTYMELSSLRSEDTA |
| | | mAb20.1 | VYYCSREDDYDGTPDAMDYWGQGTLVTVSS |
| 2 | aa | VL | DIQMTQSPSSLSASVGDRVTITCHASQNINLWLSWYQQKPGKAPKLL |
| | | Humanized | IYRASNLHTGVPSRFSGSGSATDFTFTISSLQPEDIATYYCQQGHSYPY |
| | | mAb 20.1 | TFGGGTKVDIK |
| 3 | aa | VH | QVQLVQSGAEVKKPGASVKLSCKASGYIFTRYYMYWVKQRPGQGL |
| | | Humanized | EWIGEINPNNGGTKFNEKFKNRATLTVDKSISTAYMELSRLRSDDTA |
| | | mAb 7.2 | VYYCSREDDYDGTPFAMDYWGQGTLVTVSS |
| 4 | aa | VL | DIQMTQSPSSLSASVGDRVTITCHASQNINVWLSWYQQKPGKAPKLL |
| | | Humanized | IYKASNLHTGVPSRFTGSGSGTDFTFTISSLQPEDIATYYCQQGQTYP |
| | | mAb 7.2 | YTFGQGTKLEIK |
| 5 | aa | HCDR 1 | RYYLY |
| | | Humanized | |
| | | mAb20.1 | |
| 6 | aa | HCDR2 | EINPNNGGTKFNEKFKS |
| | | Humanized | |
| | | mAb20.1 | |
| 7 | aa | HCDR3 | EDDYDGTPDAMDY |
| | | Humanized | |
| | | mAb20.1 | |
| 8 | aa | LCDR1 | HASQNINLWLS |
| | | Humanized | |
| | | mAb20.1 | |
| 9 | aa | LCDR2 | RASNLHT |
| | | Humanized | |
| | | mAb20.1 | |
| 10 | aa | LCDR3 | QQGHSYPYT |
| | | Humanized | |
| | | mAb20.1 | |
| 11 | aa | HCDR 1 | RYYMY |
| | | Humanized | |
| | | mAb7.2 | |
| 12 | aa | HCDR2 | EINPNNGGTKFNEKFKN |
| | | Humanized | |
| | | mAb7.2 | |
| 13 | aa | HCDR3 | EDDYDGTPFAMDY |
| | | Humanized | |
| | | mAb7.2 | |
| 14 | aa | LCDR1 | HASQNINVWLS |
| | | Humanized | |
| | | mAb7.2 | |
| 15 | aa | LCDR2 | KASNLHT |
| | | Humanized | |
| | | mAb7.2 | |
| 16 | aa | LCDR3 | QQGQTYPYT |
| | | Humanized | |
| | | mAb7.2 | |
| 17 | aa | HCDR1 | SYLIH |
| | | Humanized | |
| | | mAb103.2 | |
| 18 | aa | HCDR2 | VINPRSGDSHYNEKFKD |
| | | Humanized | |
| | | mAb103.2 | |
| 19 | aa | HCDR3 | SDYGAY |
| | | Humanized | |
| | | mAb103.2 | |
| 20 | aa | LCDR1 | RASQSISNNLH |
| | | Humanized | |
| | | mAb103.2 | |
| 21 | aa | LCDR2 | YASQSAF |
| | | Humanized | |
| | | mAb103.2 | |
| 22 | aa | LCDR3 | QQSNSWPHT |
| | | Humanized | |
| | | mAb103.2 | |
| 23 | aa | Heavy Chain | QVQLVQSGAEVKKPGASVKVSCKASGYTFTRYYLYWVKQRPGQGL |
| | | Humanized | EWIGEINPNNGGTKFNEKFKSRATMTVDKSTRTTYMELSSLRSEDTA |
| | | mAb20.1 | VYYCSREDDYDGTPDAMDYWGQGTLVTVSSASTKGPSVFPLAPSSK |
| | | | STSGGTAALGCLVKDYFPEPVTVSWNSGALTSGVHTFPAVLQSSGL |
| | | | YSLSSVVTVPSSSLGTQTYICNVNHKPSNTKVDKRVEPKSCDKTHTC |
| | | | PPCPAPEFEGGPSVFLFPPKPKDTLMISRTPEVTCVVVDVSHEDPEVK |
| | | | FNWYVDGVEVHNAKTKPREEQYNSTYRVVSVLTVLHQDWLNGKE |
| | | | YKCKVSNKALPASIEKTISKAKGQPREPQVYTLPPSREEMTKNQVSL |
| | | | TCLVKGFYPSDIAVEWESNGQPENNYKTTPPVLDSDGSFFLYSKLTV |
| | | | DKSRWQQGNVFSCSVMHEALHNHYTQKSLSLSPGK. |
| 24 | aa | Light Chain | DIQMTQSPSSLSASVGDRVTITCHASQNINLWLSWYQQKPGKAPKLL |
| | | Humanized | IYRASNLHTGVPSRFSGSGSATDFTFTISSLQPEDIATYYCQQGHSYPY |
| | | mAb 20.1 | TFGGGTKVDIKRTVAAPSVFIFPPSDEQLKSGTASVVCLLNNFYPREA |
| | | | KVQWKVDNALQSGNSQESVTEQDSKDSTYSLSSTLTLSKADYEKHK |
| | | | VYACEVTHQGLSSPVTKSFNRGEC |
| 25 | aa | VL de Vk2 7.2 | DIQMTQSPSSLSASVGDRVTITCHASQNINVWLSWYQQKPGKAPKLL |
| | | | IYKASNLHTGVPSRFSGSGSGTDFTFTISSLQPEDIATYYCQQGQTYP |
| | | | YTFGQGTKLEIK |
| 26 | aa | Heavy Chain | QVQLVQSGAEVKKPGASVKLSCKASGYIFTRYYMYWVKQRPGQGL |
| | | Humanized | EWIGEINPNNGGTKFNEKFKNRATLTVDKSISTAYMELSRLRSDDTA |
| | | mAb 7.2 | VYYCSREDDYDGTPFAMDYWGQGTLVTVSSASTKGPSVFPLAPSSK |
| | | (mAb 1) | STSGGTAALGCLVKDYFPEPVTVSWNSGALTSGVHTFPAVLQSSGL |
| | | | YSLSSVVTVPSSSLGTQTYICNVNHKPSNTKVDKRVEPKSCDKTHTC |
| | | | PPCPAPEFEGGPSVFLFPPKPKDTLMISRTPEVTCVVVDVSHEDPEVK |
| | | | FNWYVDGVEVHNAKTKPREEQYNSTYRVVSVLTVLHQDWLNGKE |
| | | | YKCKVSNKALPASIEKTISKAKGQPREPQVYTLPPSREEMTKNQVSL |
| | | | TCLVKGFYPSDIAVEWESNGQPENNYKTTPPVLDSDGSFFLYSKLTV |
| | | | DKSRWQQGNVFSCSVMHEALHNHYTQKSLSLSPG |
| 27 | aa | Light chain | DIQMTQSPSSLSASVGDRVTITCHASQNINVWLSWYQQKPGKAPKLL |
| | | humanized | IYKASNLHTGVPSRFTGSGSGTDFTFTISSLQPEDIATYYCQQGQTYP |
| | | mAb 7.2 | YTFGQGTKLEIKRTVAAPSVFIFPPSDEQLKSGTASVVCLLNNFYPRE |
| | | (mAb 1) | AKVQWKVDNALQSGNSQESVTEQDSKDSTYSLSSTLTLSKADYEKH |
| | | | KVYACEVTHQGLSSPVTKSFNRGEC |
| 28 | nt | Coding VH3 | CAGGTCCAACTGGTGCAGTCTGGGGCTGAAGTGAAGAAGCCTGG |
| | | humanized | GGCTTCAGTGAAGGTTTCCTGCAAGGCTTCTGGCTACACCTTCAC |
| | | mAb 20.1 | TCGGTACTATTTGTACTGGGTGAAACAGAGGCCTGGACAAGGCC |
| | | (mAb 3) | TTGAGTGGATTGGAGAGATAAATCCTAACAATGGTGGTACTAA |
| | | | GTTCAATGAGAAGTTCAAGAGCAGAGCCACAATGACTGTAGAC |
| | | | AAATCCACGAGAACAACATACATGGAGCTCAGCAGCCTGAGATC |
| | | | TGAGGACACGGCGGTCTATTACTGTTCAAGAGAGGATGATTACG |
| | | | ACGGGACCCCCGATGCTATGGACTACTGGGGTCAAGGAACCCT |
| | | | GGTCACCGTCTCCTCA |
| 29 | nt | Coding Vk1 | GACATCCAGATGACCCAGTCTCCATCCAGTCTGTCTGCATCCGTA |
| | | humanized | GGAGACAGAGTCACCATCACTTGCCATGCCAGTCAGAACATTA |
| | | mAb 20.1 | ATCTTTGGTTATCTTGGTACCAGCAGAAACCAGGAAAAGCCCCT |
| | | (mAb 3) | AAACTTCTGATCTATAGGGCTTCCAACTTGCACACAGGCGTCCC |
| | | | ATCAAGGTTTAGTGGCAGTGGATCTGCAACAGATTTCACATTCAC |
| | | | CATCAGCAGCCTGCAGCCTGAAGACATTGCCACTTACTACTGTCA |
| | | | ACAGGGTCATAGTTATCCGTACACGTTCGGAGGGGGGACCAAA |
| | | | GTGGATATCAAA |
| 30 | nt | Coding heavy | CAGGTCCAACTGGTGCAGTCTGGGGCTGAAGTGAAGAAGCCTGG |
| | | chain | GGCTTCAGTGAAGTTGTCCTGCAAGGCTTCTGGCTACATCTTCAC |
| | | humanized | CAGATACTATATGTATTGGGTGAAGCAGAGGCCTGGACAAGGCC |
| | | mAb 7.2 | TTGAGTGGATTGGAGAGATTAATCCTAACAATGGTGGTACTAAGT |
| | | | TCAATGAGAAGTTCAAGAACAGGGCCACACTGACTGTAGACAAA |
| | | | TCCATCAGCACAGCATACATGGAGCTCAGCAGGCTGAGATCTGA |
| | | | CGACACGGCGGTCTATTATTGTTCAAGAGAGGATGATTACGACGG |
| | | | GACCCCCTTTGCTATGGACTACTGGGGTCAAGGAACCCTGGTCAC |
| | | | CGTCTCCTCAGCCTCCACCAAGGGCCCATCGGTCTTCCCCCTGGC |
| | | | ACCCTCCTCCAAGAGCACCTCTGGGGGCACAGCGGCCCTGGGCTG |
| | | | CCTGGTCAAGGACTACTTCCCCGAACCGGTGACGGTGTCGTGGAA |
| | | | CTCAGGCGCCCTGACCAGCGGCGTGCACACCTTCCCGGCTGTCCT |
| | | | ACAGTCCTCAGGACTCTACTCCCTCAGCAGCGTGGTGACCGTGCC |
| | | | CTCCAGCAGCTTGGGCACCCAGACCTACATCTGCAACGTGAATCA |
| | | | CAAGCCCAGCAACACCAAGGTGGACAAGAGAGTTGAGCCCAAAT |
| | | | CTTGTGACAAAACTCACACATGCCCACCGTGCCCAGCACCTGAAT |
| | | | TCGAGGGGGGACCGTCAGTCTTCCTCTTCCCCCCAAAACCCAAGG |
| | | | ACACCCTCATGATCTCCCGGACCCCTGAGGTCACATGCGTGGTGG |
| | | | TGGACGTGAGCCACGAAGACCCTGAGGTCAAGTTCAACTGGTAC |
| | | | GTGGACGGCGTGGAGGTGCATAATGCCAAGACAAAGCCGCGGGA |
| | | | GGAGCAGTACAACAGCACGTACCGTGTGGTCAGCGTCCTCACCGT |
| | | | CCTGCACCAGGACTGGCTGAATGGCAAGGAGTACAAGTGCAAGG |
| | | | TCTCCAACAAAGCCCTCCCAGCCTCCATCGAGAAAACCATCTCCA |
| | | | AAGCCAAAGGGCAGCCCCGAGAACCACAGGTGTACACCCTGCCC |
| | | | CCATCCCGGGAAGAGATGACCAAGAACCAGGTCAGCCTGACCTG |
| | | | CCTGGTCAAAGGCTTCTATCCCAGCGACATCGCCGTGGAGTGGGA |
| | | | GAGCAATGGGCAGCCGGAGAACAACTACAAGACCACGCCTCCCG |
| | | | TGCTGGACTCCGACGGCTCCTTCTTCCTCTATAGCAAGCTCACCGT |
| | | | GGACAAGAGCAGGTGGCAGCAGGGGAACGTCTTCTCATGCTCCG |
| | | | TGATGCATGAGGCTCTGCACAACCACTACACGCAGAAGAGCCTCT |
| | | | CCCTGTCTCCGGGTTGA |
| 31 | nt | Coding light | GACATCCAGATGACCCAGTCTCCATCCAGTCTGTCTGCATCCGTA |
| | | chain | GGAGACAGAGTCACCATCACTTGCCATGCCAGTCAGAACATTAAT |
| | | humanized | GTTTGGTTATCTTGGTACCAGCAGAAACCAGGAAAAGCCCCTAAA |
| | | mAb 7.2 | CTCTTGATCTATAAGGCTTCCAACTTGCACACAGGCGTCCCATCA |
| | | | AGATTTACTGGCAGTGGATCTGGAACAGATTTCACATTCACCATC |
| | | | AGCAGCCTGCAGCCTGAAGACATTGCCACTTACTACTGTCAACAG |
| | | | GGTCAAACTTATCCATACACGTTCGGACAGGGGACCAAGCTGGA |
| | | | GATCAAACGAACTGTGGCTGCACCATCTGTCTTCATCTTCCCGCC |
| | | | ATCTGATGAGCAGTTGAAATCTGGAACTGCCTCTGTTGTGTGCCT |
| | | | GCTGAATAACTTCTATCCCAGAGAGGCCAAAGTACAGTGGAAGG |
| | | | TGGATAACGCCCTCCAATCGGGTAACTCCCAGGAGAGTGTCACA |
| | | | GAGCAGGACAGCAAGGACAGCACCTACAGCCTCAGCAGCACCCT |
| | | | GACGCTGAGCAAAGCAGACTACGAGAAACACAAAGTCTACGCCT |
| | | | GCGAAGTCACCCATCAGGGCCTGAGCTCGCCCGTCACAAAGAGC |
| | | | TTCAACAGGGGAGAGTGTTAG |
| 32 | aa | Human | MKMASFLAFLLLNFRVCLLLLQLLMPHSAQFSVLGPSGPILAMVGED |
| | | BTN3A1 | ADLPCHLFPTMSAETMELKWVSSSLRQVVNVYADGKEVEDRQSAP |
| | | | YRGRTSILRDGITAGKAALRIHNVTASDSGKYLCYFQDGDFYEKALV |
| | | | ELKVAALGSDLHVDVKGYKDGGIHLECRSTGWYPQPQIQWSNNKG |
| | | | ENIPTVEAPVVADGVGLYAVAASVIMRGSSGEGVSCTIRSSLLGLEK |
| | | | TASISIADPFFRSAQRWIAALAGTLPVLLLLLGGAGYFLWQQQEEKK |
| | | | TQFRKKKREQELREMAWSTMKQEQSTRVKLLEELRWRSIQYASRGE |
| | | | RHSAYNEWKKALFKPADVILDPKTANPILLVSEDQRSVQRAKEPQD |
| | | | LPDNPERFNWHYCVLGCESFISGRHYWEVEVGDRKEWHIGVCSKNV |
| | | | QRKGWVKMTPENGFWTMGLTDGNKYRTLTEPRTNLKLPKPPKKVG |
| | | | VFLDYETGDISFYNAVDGSHIHTFLDVSFSEALYPVFRILTLEPTALTI |
| | | | CPA |
| 33 | aa | Human | MKMASSLAFLLLNFHVSLLLVQLLTPCSAQFSVLGPSGPILAMVGED |
| | | BTN3A2 | ADLPCHLFPTMSAETMELKWVSSSLRQVVNVYADGKEVEDRQSAP |
| | | | YRGRTSILRDGITAGKAALRIHNVTASDSGKYLCYFQDGDFYEKALV |
| | | | ELKVAALGSNLHVEVKGYEDGGIHLECRSTGWYPQPQIQWSNAKGE |
| | | | NIPAVEAPVVADGVGLYEVAASVIMRGGSGEGVSCIIRNSLLGLEKT |
| | | | ASISIADPFFRSAQPWIAALAGTLPILLLLLAGASYFLWRQQKEITALS |
| | | | SEIESEQEMKEMGYAATEREISLRESLQEELKRKKIQYLTRGEESSSD |
| | | | TNKSA |
| 34 | aa | Human | MKMASSLAFLLLNFHVSLFLVQLLTPCSAQFSVLGPSGPILAMVGED |
| | | BTN3A3 | ADLPCHLFPTMSAETMELRWVSSSLRQVVNVYADGKEVEDRQSAP |
| | | | YRGRTSILRDGITAGKAALRIHNVTASDSGKYLCYFQDGDFYEKALV |
| | | | ELKVAALGSDLHIEVKGYEDGGIHLECRSTGWYPQPQIKWSDTKGE |
| | | | NIPAVEAPVVADGVGLYAVAASVIMRGSSGGGVSCIIRNSLLGLEKT |
| | | | ASISIADPFFRSAQPWIAALAGTLPISLLLLAGASYFLWRQQKEKIALS |
| | | | RETEREREMKEMGYAATEQEISLREKLQEELKWRKIQYMARGEKSL |
| | | | AYHEWKMALFKPADVILDPDTANAILLVSEDQRSVQRAEEPRDLPD |
| | | | NPERFEWRYCVLGCENFTSGRHYWEVEVGDRKEWHIGVCSKNVER |
| | | | KKGWVKMTPENGYWTMGLTDGNKYRALTEPRTNLKLPEPPRKVGI |
| | | | FLDYETGEISFYNATDGSHIYTFPHASFSEPLYPVFRILTLEPTALTICPI |
| | | | PKEVESSPDPDLVPDHSLETPLTPGLANESGEPQAEVTSLLLPAHPGA |
| | | | EVSPSATTNQNHKLQARTEALY |
| 35 | nt | ACTB | GGAAATCGTGCGTGACATTA |
| | | forward | |
| | | primer | |
| 36 | nt | ACTB | AGGAGGAAGGCTGGAAGAG |
| | | reverse | |
| | | primer | |
| 37 | nt | BTN3A1 | TTCCAGGTCATAGTGTCTGC |
| | | forward | |
| | | primer | |
| 38 | nt | BTN3A1 | TGAGCAGCTGAGCAAAAGG |
| | | reverse | |
| | | primer | |
| 39 | nt | BTN3A2 | TGGGAATACCAAGGGA |
| | | forward | |
| | | primer | |
| 40 | nt | BTN3A2 | AGTGAGCAGCTGGACCAAGA |
| | | reverse | |
| | | primer | |
| 41 | nt | BTN3A3 | GAGGGAATACTAAGAAATGGT |
| | | forward | |
| | | primer | |
| 42 | nt | BTN3A3 | GAAGAGGGAGACATGAAAGT |
| | | reverse | |
| | | primer | |
| 43 | nt | COM-1 | GCACTATTTTTAGCCG-GAACCTT |
| | | forward | |
| | | primer | |
| 44 | nt | COM-1 | TTGAGGAGAAAAACTGGATTGAGA |
| | | reverse | |
| | | primer | |
| 45 | nt | TNF | AGGAGAAGAGGCTGAGGAACAAG |
| | | forward | |
| | | primer | |
| 46 | nt | TNF | GAGGGAGAGAAGCAACTACAGACC |
| | | reverse | |
| | | primer | |
| 47 | nt | IL1B | CAGCACCTCTCAAGCAGAAAAC |
| | | forward | |
| | | primer | |
| 48 | nt | IL1B | GTTGGGCATTGGTGTAGACAAC |
| | | reverse | |
| | | primer | |
| 49 | nt | IL6 | CCAGGAGAAGATTCCAAAGATG |
| | | forward | |
| | | primer | |
| 50 | nt | IL6 | GGAAGGTTCAGGTTGTTTTCTG |
| | | reverse | |
| | | primer | |
| 51 | nt | IFNG | GTTTTGGGTTCTCTTGGCTGTTA |
| | | forward | |
| | | primer | |
| 52 | nt | IFNG | ACACTCTTTTGGATGCTCTGGTC |
| | | reverse | |
| | | primer | |
| 53 | nt | CXCL10 | TCCCATCTTCCAAGGGTACTAA |
| | | forward | |
| | | primer | |
| 54 | nt | CXCL10 | GGTAGCCACTGAAAGAATTTGG |
| | | reverse | |
| | | primer | |
| 55 | nt | IL10 | GGGGGTTGAGGTATCAGAGGTAA |
| | | forward | |
| | | primer | |
| 56 | nt | IL10 | GCTCCAAGAGAAAGGCATCTACA |
| | | reverse | |
| | | primer | |
| 57 | nt | TGFB | GACATCAAAAGATAACCACTC |
| | | forward | |
| | | primer | |
| 58 | nt | TGFB | TCTATGACAAGTTCAAGCAGA |
| | | reverse | |
| | | primer | |
| 59 | nt | IL1RA | TCTATCACCAGACTTGACACA |
| | | forward | |
| | | primer | |
| 60 | nt | IL1RA | CCTAATCACTCTCCTCCTCTTCC |
| | | reverse | |
| | | primer | |
| 61 | nt | CD163 | CGGTCTCTGTGATTTGTAACCAG |
| | | forward | |
| | | primer | |
| 62 | nt | CD163 | TACTATGCTTTCCCCATCCATC |
| | | reverse | |
| | | primer | |
| 63 | aa | Humanized | QVQMQQSGAEVKKPGASVKVSCKASGYAFTSYLIHWIKQRPGQGLE |
| | | VH of 103.2 | WIGVINPRSGDSHYNEKFKDRVTMTADQSISTAYMELSRLRSDDTA |
| | | | VYYCARSDYGAYWGQGTLVTVSS |
| 64 | aa | Humanized | EIVLTQSPATLSVSPGERATLSCRASQSISNNLHWYQQKPGQAPRLLI |
| | | VL of 103.2 | KYASQSAFGIPARFSGSGSGTEFTLTISSLQSEDFAVYFCQQSNSWPH |
| | | | TFGQGTKLEIK |

### REFERENCES

Ausubel, F. M., Brent, R., Kingston, R. E., Moore, D. D., Seidman, J. G., and Struhl, K. (1988). Current Protocols in Molecular Biology (John Wiley & Sons).
Baldwin, C. L., and J. C. Telfer. 2015. Mol. Immunol. 66:35-47. doi: 10.1016/j.molimm.2014.10.024.
Baudino, L., Shinohara, Y., Nimmerjahn, F., Furukawa, J.-I., Nakata, M., Martínez-Soria, E., Petry, F., Ravetch, J. V., Nishimura, S.-I., and Izui, S. (2008). J. Immunol. 181, 6664-6669.
Bird, R. E., Hardman, K. D., Jacobson, J. W., Johnson, S., Kaufman, B. M., Lee, S. M., Lee, T., Pope, S. H., Riordan, G. S., and Whitlow, M. (1988). Science 242, 423-426.
Blazquez, J.-L., A. Benyamine, C. Pasero, and D. Olive. 2018. Front. Immunol. 9. doi: 10.3389/fimmu.2018.01601.
Bryson, C. J., Jones, T. D., and Baker, M. P. (2010). Prediction of Immunogenicity of Therapeutic Proteins. BioDrugs 24, 1-8.
Chen, S., H. Feng, H. Xu, S. Huang, J. Sun, L. Zhou, J. He, W. Song, R. Wang, X. Li, and M. Fang. 2020. Front. Med. 0. doi: 10.3389/fmed.2020.567296.
Dong, P., X. Ju, Y. Yan, S. Zhang, M. Cai, H. Wang, H. Chen, Y. Hu, L. Cui, J. Zhang, and W. He. 2018. Front. Immunol. 9. doi: 10.3389/fimmu.2018.02812.
Goeddel, D. V. (1990). [1] Systems for heterologous gene expression. In Methods in Enzymology, (Academic Press), pp. 3-7.
Harly, C., Y. Guillaume, S. Nedellec, C.-M. Peigné, H. Monkkönen, J. Monkkönen, J. Li, J. Kuball, E. J. Adams, S. Netzer, J. Déchanet-Merville, A. Léger, T. Herrmann, R. Breathnach, D. Olive, M. Bonneville, and E. Scotet. 2012. Blood. 120:2269-2279. doi: 10.1182/blood-2012-05-430470.
Huston, J. S., Levinson, D., Mudgett-Hunter, M., Tai, M. S., Novotný, J., Margolies, M. N., Ridge, R. J., Bruccoleri, R. E., Haber, E., and Crea, R. (1988). Proc. Natl. Acad. Sci. 85, 5879-5883.
Kabat, E. A., Wu, T. T., Foeller, C., Perry, H. M., and Gottesman, K. S. (1992). Sequences of Proteins of Immunological Interest (DIANE Publishing).
Kaufman, R. J., and Sharp, P. A. (1982). Mol. Cell. Biol. 2, 1304-1319.
Lei, L., H. Qian, X. Yang, X. Zhang, D. Zhang, T. Dai, R. Guo, L. Shi, Y. Cheng, B. Zhang, X. Zhou, J. Hu, and Y. Guo. 2020. J. Cell. Mol. Med. 24:11603-11606. doi: https://doi.org/10.1111/jcmm.15620.
McCafferty, J., Griffiths, A. D., Winter, G., and Chiswell, D. J. (1990). Nature 348, 552-554.
Morrison, S. L. (1985). Science 229, 1202-1207.
Oganesyan, V., Gao, C., Shirinian, L., Wu, H., and Dall'Acqua, W. F. (2008). Acta Crystallogr. D Biol. Crystallogr. 64, 700-704.
Perry, L. C. A., Jones, T. D., and Baker, M. P. (2008). Drugs RD 9, 385-396.
Poccia, F., C. Agrati, C. Castilletti, L. Bordi, C. Gioia, D. Horejsh, G. Ippolito, P. K. S. Chan, D. S. C. Hui, J. J. Y. Sung, M. R. Capobianchi, and M. Malkovsky. 2006. J. Infect. Dis. 193:1244-1249. doi:10.1086/502975.
Poccia, F., C. Agrati, F. Martini, G. Mejia, M. Wallace, and M. Malkovsky. 2005. Immunol. Lett. 100:14-20. doi: 10.1016/j.imlet.2005.06.025.
Qin, G., H. Mao, J. Zheng, S. F. Sia, Y. Liu, P.-L. Chan, K.-T. Lam, J. S. M. Peiris, Y.-L. Lau, and W. Tu. 2009. J. Infect. Dis. 200: 858-865. doi: 10.1086/605413.
Reddy, M. P., Kinney, C. A. S., Chaikin, M. A., Payne, A., Fishman-Lobell, J., Tsui, P., Monte, P. R. D., Doyle, M. L., Brigham-Burke, M. R., Anderson, D., et al. (2000). J. Immunol. 164, 1925-1933.
Remington, J. P., and Gennaro, A. R. (1995). Remington: the science and practice of pharmacy (Easton, PA: Mack Publishing).
Rijkers, G., T. Vervenne, and P. van der Pol. 2020. Cell. Mol. Immunol. 17:771-772. doi:10.1038/s41423-020-0473-0.
Alegre, M.-L., Frauwirth, K. A., and Thompson, C. B. (2001). Nat. Rev. Immunol. 1, 220-228.
Ausubel, F. M., Brent, R., Kingston, R. E., Moore, D. D., Seidman, J. G., and Struhl, K. (1988). Current Protocols in Molecular Biology (John Wiley & Sons).
Baudino, L., Shinohara, Y., Nimmerjahn, F., Furukawa, J.-I., Nakata, M., Martinez-Soria, E., Petry, F., Ravetch, J. V., Nishimura, S.-I., and Izui, S. (2008). J. Immunol. 181, 6664-6669.
Bensussan, A., and Olive, D. (2005). T-cell: Section report. Cell. Immunol. 236, 3-5.
Bird, R. E., Hardman, K. D., Jacobson, J. W., Johnson, S., Kaufman, B. M., Lee, S. M., Lee, T., Pope, S. H., Riordan, G. S., and Whitlow, M. (1988). Science 242, 423-426.
Brennan, M., Davison, P. F., and Paulus, H. (1985). Science 229, 81-83.
Bryson, C. J., Jones, T. D., and Baker, M. P. (2010). BioDrugs 24, 1-8.
Chapoval, A. I., Ni, J., Lau, J. S., Wilcox, R. A., Flies, D. B., Liu, D., Dong, H., Sica, G. L., Zhu, G., Tamada, K., et al. (2001). Nat. Immunol. 2, 269-274.
Collins, M., Ling, V., and Carreno, B. M. (2005). Genome Biol. 6, 223.
Coyle, A. J., and Gutierrez-Ramos, J. C. (2001). Nat. Immunol. 2, 203-209.
Dong, H., Zhu, G., Tamada, K., and Chen, L. (1999). Nat. Med. 5, 1365-1369.
Freeman, G. J., Long, A. J., Iwai, Y., Bourque, K., Chernova, T., Nishimura, H., Fitz, L. J., Malenkovich, N., Okazaki, T., Byrne, M. C., et al. (2000). J. Exp. Med. 192, 1027-1034.
Glennie, M. J., McBride, H. M., Worth, A. T., and Stevenson, G. T. (1987). J. Immunol. 139, 2367-2375.
Goeddel, D. V. (1990). [1] Systems for heterologous gene expression. In Methods in Enzymology, (Academic Press), pp. 3-7.
Gu, S., Nawrocka, W., and Adams, E. J. (2015). Front. Immunol. 5.
Harly, C., Guillaume, Y., Nedellec, S., Peigné, C.-M., Monkkönen, H., Monkkönen, J., Li, J., Kuball, J., Adams, E. J., Netzer, S., et al. (2012). Blood 120, 2269-2279.
Huston, J. S., Levinson, D., Mudgett-Hunter, M., Tai, M. S., Novotný, J., Margolies, M. N., Ridge, R. J., Bruccoleri, R. E., Haber, E., and Crea, R. (1988). Proc. Natl. Acad. Sci. 85, 5879-5883.
Kabat, E. A., Wu, T. T., Foeller, C., Perry, H. M., and Gottesman, K. S. (1992). Sequences of Proteins of Immunological Interest (DIANE Publishing).
Karpovsky, B., Titus, J. A., Stephany, D. A., and Segal, D. M. (1984). J. Exp. Med. 160, 1686-1701.
Kaufman, R. J., and Sharp, P. A. (1982). Mol. Cell. Biol. 2, 1304-1319.
Khattri, R., Auger, J. A., Griffin, M. D., Sharpe, A. H., and Bluestone, J. A. (1999). J. Immunol. 162, 5784-5791.
Klocke, K., Sakaguchi, S., Holmdahl, R., and Wing, K. (2016). Proc. Natl. Acad. Sci. 113, E2383-E2392.
Latchman, Y., Wood, C. R., Chernova, T., Chaudhary, D., Borde, M., Chernova, I., Iwai, Y., Long, A. J., Brown, J. A., Nunes, R., et al. (2001). Nat. Immunol. 2, 261-268.
Linsley, P. S., Greene, J. L., Tan, P., Bradshaw, J., Ledbetter, J. A., Anasetti, C., and Damle, N. K. (1992). J. Exp. Med. 176, 1595-1604.
Liu, M. A., Kranz, D. M., Kurnick, J. T., Boyle, L. A., Levy, R., and Eisen, H. N. (1985). Proc. Natl. Acad. Sci. 82, 8648-8652.
McCafferty, J., Griffiths, A. D., Winter, G., and Chiswell, D. J. (1990). Nature 348, 552-554.
Morrison, S. L. (1985). Science 229, 1202-1207.
Ni, L., and Dong, C. (2017). Mol. Cancer Ther. 16, 1203-1211.
Oganesyan, V., Gao, C., Shirinian, L., Wu, H., and Dall'Acqua, W. F. (2008). Acta Crystallogr. D Biol. Crystallogr. 64, 700-704.
Panowski, S., Bhakta, S., Raab, H., Polakis, P., and Junutula, J. R. (2013). MAbs 6, 34-45.
Paulus, H. (1985). Behring Inst. Mitt. 118-132.
Perry, L. C. A., Jones, T. D., and Baker, M. P. (2008). Drugs RD 9, 385-396.
Reddy, M. P., Kinney, C. A. S., Chaikin, M. A., Payne, A., Fishman-Lobell, J., Tsui, P., Monte, P. R. D., Doyle, M. L., Brigham-Burke, M. R., Anderson, D., et al. (2000). J. Immunol. 164, 1925-1933.
Remington, J. P., and Gennaro, A. R. (1995). Remington: the science and practice of pharmacy (Easton, PA: Mack Publishing).
Rhodes, D. A., Stammers, M., Malcherek, G., Beck, S., and Trowsdale, J. (2001). Genomics 71, 351-362.
Ruddy, D. A., Kronmal, G. S., Lee, V. K., Mintier, G. A., Quintana, L., Domingo, R., Meyer, N. C., Irrinki, A., McClelland, E. E., Fullan, A., et al. (1997). Genome Res. 7, 441-456.
Saverino, D., Tenca, C., Zarcone, D., Merlo, A., Megiovanni, A. M., Valle, M. T., Manca, F., Grossi, C. E., and Ciccone, E. (1999). J. Immunol. 162, 651-658.
Schneider T, Jahn H-U, Liesenfeld O, et al. Clin Infect Dis 1997; 24:261-4.
Sharpe, A. H., and Freeman, G. J. (2002). Nat. Rev. Immunol. 2, 116-126.
Sharpe, A. H., and Pauken, K. E. (2018). Nat. Rev. Immunol. 18, 153-167.
Shukla, A. A., Hubbard, B., Tressel, T., Guhan, S., and Low, D. (2007). J. Chromatogr. B 848, 28-39.
Stech, M., Nikolaeva, O., Thoring, L., Stöcklein, W. F. M., Wüstenhagen, D. A., Hust, M., Dübel, S., and Kubick, S. (2017). Sci. Rep. 7, 12030.
Strohl, W. R. (2009). Curr. Opin. Biotechnol. 20, 685-691.
Takebe, Y., Seiki, M., Fujisawa, J., Hoy, P., Yokota, K., Arai, K., Yoshida, M., and Arai, N. (1988). Mol. Cell. Biol. 8, 466-472.
Urlaub, G., and Chasin, L. A. (1980). Proc. Natl. Acad. Sci. U.S.A. 77, 4216-4220.
Yazdanifar, M., N. Mashkour, and A. Bertaina. 2020. Crit. Rev. Microbiol. 46:689-702. doi:10.1080/1040841X.2020.1822279.

1. A method for treating an infectious disorder in a human subject in need thereof comprising administering a therapeutically efficient amount of an anti-BTN3A activating antibody, in said subject.

2. The method of claim 1, wherein said BTN3A activating antibody has one or more of the following properties:
(i) it binds to human PBMCs with an EC₅₀ of 50 μg/ml or below, as measured in a flow cytometry assay;
(ii) it induces in vitro the activation of γδ-T cells, in co-culture with BTN3A expressing cells, with an EC₅₀ below 5 μg/ml, as measured with a degranulation assay;
(iii) it potentiates in vitro the reduction of C. burnetii bacterial load, as measured in vitro with co-cultures of monocytes in the presence of Vγ9Vδ2 T cells;
(iv) it increases in vitro cytotoxic activity of Vγ9Vδ2 T cells towards burnetii infected cells, as measured in vitro with co-cultures of infected monocytes; and/or
it increases in vitro cytotoxic activity of Vγ9Vδ2 T cells towards SARS-Cov2 infected cells as measured in vitro in co-cultures of infected cells with Vγ9Vδ2 T cells.

3. (canceled)

4. The method of claim 1, wherein said BTN3A activating antibody either:
(i) comprises HCDRs1-3 of SEQ ID NO:5-7 and LCDRs1-3 of SEQ ID NO: 8-10;
(ii) comprises HCDRs1-3 of SEQ ID NO:11-13 and LCDRs1-3 of SEQ ID NO:14-16;
(iii) comprises HCDRs1-3 of SEQ ID NO:17-19 and LCDRs1-3 of SEQ ID NO:20-22;
(iv) comprises a variable heavy chain (VH) polypeptide comprising an amino acid sequence that is at least about 95%, 96%, 97%, 98%, 99% or 100% identical to the amino acid sequence of SEQ ID NO:1, and a variable light chain (VL) polypeptide comprising an amino acid sequence that is at least about 95%, 96%, 97%, 98%, 99% or 100% identical to of SEQ ID NO:-2;
(v) comprises a variable heavy chain (VH) polypeptide comprising an amino acid sequence that is at least about 95%, 96%, 97%, 98%, 99% or 100% identical to the amino acid sequence of SEQ ID NO:3, and a variable light chain (VL) polypeptide comprising an amino acid sequence that is at least about 95%, 96%, 97%, 98%, 99% or 100% identical to of SEQ ID NO: 4;
(vi) comprises a heavy chain of SEQ ID NO: 23 and a light chain of SEQ ID NO: 24;
(vii) competes for binding with mAb 20.1 as produced by the hybridoma deposited at the CNCM under deposit number I-4401, or
(viii) competes for binding with mAb 7.2 as produced by the hybridoma deposited at the CNCM under deposit number I-4402.

5. (canceled)

6. The method of claim 1, wherein said BTN3A activating antibody comprises a mutant or chemically modified IgG1 constant region, wherein said mutant or chemically modified IgG1 constant region confers no or decreased binding to Fcγ receptors when compared to a corresponding antibody with wild type IgG1 isotype constant region.

7. (canceled)

8. The method of claim 1, for treating a disorder caused by SARS-Cov2 infection, wherein: said BTN3A activating antibody is administered in combination, simultaneously or separately with a second drug substance selected from an antiviral treatment, an anti-inflammatory treatment and a cell therapy product.

9. (canceled)

10. (canceled)

11. (canceled)

12. The method of claim 9, for treating a disorder caused by Coxiella burnetii infection, wherein said BTN3A antibody is administered in combination, simultaneously or separately with a second drug substance selected from an antibacterial treatment and a cell therapy product.

13. (canceled)

14. (canceled)

15. The method of claim 1, wherein said BTN3A activating antibody is administered to the subject in need thereof, by intravenous infusion.

16. An isolated BTN3A activating antibody comprising a variable heavy chain polypeptide VH at least 95% identical to SEQ ID NO:1 and a variable light chain polypeptide VL at least 95% to SEQ ID NO: 2 and comprising HCDRs 1-3 of SEQ ID NO: 5-7 and LCDRs 1-3 of SEQ ID NO: 8-10.

17. The BTN3A activating antibody of claim 16, comprising a variable heavy chain polypeptide VH of SEQ ID NO: 1 and a variable light chain polypeptide VL of SEQ ID NO: 2.

18. The BTN3A activating antibody of claim 16, comprising a heavy chain polypeptide of SEQ ID NO: 23 and a light chain polypeptide of SEQ ID NO: 24.

19. A nucleic acid molecule encoding the heavy and light chains of a BTN3A activating antibody according to claim 16.

20. An expression vector comprising at least one nucleic acid according to claim 19.

21. A host cell comprising an expression vector according to claim 20.

22. A pharmaceutical composition comprising an anti-BTN3A antibody according to any one of claim 16, in combination with one or more of a pharmaceutically acceptable excipient, diluent or carrier.

23. Use of the BTN3A activating antibody of claim 16.

24. The method of claim 1, wherein said infectious disorder is selected from the group consisting of (i) disorder caused by SARS-Cov2 infection, and (ii) disorder caused by Coxiella burnetii infection.

25. The method of claim 8, wherein said an antiviral or anti-inflammatory treatment is selected from the group consisting of remdesivir, baricitinib, bamlanivimab, bamlanivimab/etesevimab, casirivimab/imdevimab, dexamethasone, budesonide and tocilizumab.

26. The method of claim 1, for treating a disorder caused by SARS-Cov2 infection, wherein said subject is a human subject which has been diagnosed as being SARS-Cov2 positive, is a human subject which has mild or moderate COVID-19, is a subject which is at high risk of progressing to severe COVID-19, is a subject having severe COVID-19, and/or is a subject having severe COVID-19.

27. The method of claim 12, wherein said antibacterial treatment is selected from doxycycline, tetracycline, chloramphenicol, ciprofloxacin, ofloxacin, and hydroxychloroquine.

28. The method of claim 1 for treating a disorder caused by Coxiella burnetii infection, wherein said subject has been diagnosed as being positive for Coxiella burnetii infection and/or wherein said subject has Q fever.
